SEQUENCE LISTING (4 sequences)

SEQ ID NO: 1 - DNA, 23 bp, Artificial Sequence
  Primer
aaggacatcagccagaacaagcg

SEQ ID NO: 2 - DNA, 24 bp, Artificial Sequence
  Primer
aagaagtcctgcagcagcttctgc

SEQ ID NO: 3 - DNA, 24 bp, Artificial Sequence
  Primer
aaggtcggagtcaacggatttggt

SEQ ID NO: 4 - DNA, 24 bp, Artificial Sequence
  Primer
catggttcacacccatgacgaaca

(19) United States
(12) Patent Application Publication
(10) Pub. No.: US 2009/0093538 A1
(43) Pub. Date: Apr. 9, 2009

(54) Method for treating cancer

(75) Inventors: John Bertin, Winchester, MA (US); Jessica R. Kirshner, New York, NY (US); Zhenjian Du, Northborough, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Synta Pharmaceuticals Corp, Lexington, MA (US)

(21) Appl. No.: 12/005,654

(22) Filed: Dec. 27, 2007

Related U.S. Application Data
(60) Provisional application No. 60/878,557, filed on Jan. 3, 2007.

(51) Int. Cl. A61K 31/337; C12Q 1/68; A61K 31/341; A61K 31/00; A61K 31/165; A61P 35/00

(52) U.S. Cl. 514/461; 514/449; 435/6; 514/1; 514/599

(57) ABSTRACT

The invention relates to a method of treating cancer in a subject, comprising administering to the subject an anti-cancer therapy and a compound that increases the oxidative stress of the cancer cells and activates p38.

### RELATED APPLICATION

This application claims the benefit of U.S. Provisional Application No. 60/878,557, filed on Jan. 3, 2007, the entire teachings of which are incorporated herein by reference.

### BACKGROUND OF THE INVENTION

Radiation therapy and chemotherapy are commonly used treatments for cancer. However, it is well known that these therapies often result in incomplete killing of the cancer cells and that reoccurrence of the cancer can result in cancer that is more resistant to radiation or chemotherapy.

Many chemotherapeutic drugs cause cells to generate reactive oxygen species (ROS) which can lead to cellular signaling that induces either apoptosis or proliferation. ROS are generated during normal metabolism in the mitochondria of cells. However, the buffering action of endogenous thiols, such as glutathione and thioredoxin, protect cells from oxidative damage and help maintain the reduction-oxidation (redox) state of the cell. If ROS are elevated to a level which exceeds the buffering capacity of the cell, activation of signaling pathways and gene expression that induce apoptosis can occur. Cancer cells are generally more susceptible to ROS induced apoptosis than normal cells because the environment created by uncontrolled growth of a tumor is typically hypoxic. However, radiation therapy and chemotherapy that results in incomplete killing of cancer cells can result in the remaining cancer cells developing a resistance to oxidative stress. For example, cancer cells can compensate for the generation of ROS by upregulating the amount of glutathione (GSH) or components of the thioredoxin system, such as thioredoxin reductase. GSH is a cellular antioxidant that contains a reducing thiol group which

donates electrons to ROS, thereby neutralize them. The thioredoxin system includes thioredoxin, a protein that has a redox-active disulfide group which can be reduced by thioredoxin reductase to two dithiol groups in the presence of NADPH. The dithiol form of thioredoxin is a powerful protein-disulfide reductase which helps regulate the redox state of the cell. Cellular resistance to anticancer drugs such as paclitaxel has been shown to be proportional to the total antioxidant capacity of a target cell.

By upregulating cellular defense mechanisms against ROS, cancer cells can evade destruction by radiation and chemotherapeutic drugs. Therefore, a need exists for agents that reduce cellular mechanism of compensating for ROS and induce cellular signaling pathways that lead to apoptosis. Such agents would be expected to increase the effectiveness of existing anti-cancer agents.

### SUMMARY OF THE INVENTION

The present invention relates to the discovery that the efficacy of standard treatments for cancer, such as chemotherapy or radiation treatment, can be increased by administering them in combination with an agent that increase the oxidative stress of cancer cells by inhibiting the mechanisms that cancer cells utilize to compensate for ROS and/or activating cellular signaling pathways that lead to immunocytotoxicity.

In one aspect, the invention relates to a compound that increases oxidative stress in a cell, wherein the compound increases p38 activity. p38 is a member of the mitogen-activated protein (MAP) kinase family. MAP kinases are signaling pathways that respond to extracellular stimuli and can be activated by ROS. The MAP kinase family has four distinct subgroups: 1) extracellular signal-regulated kinases (ERK); 2) c-jun N-terminal or stress-activated protein kinases (JNK/SAPK); 3) ERK/big MAP kinase 1 (BMK1); and the p38 group of protein kinases. In one embodiment, the compound that increases oxidative stress in a cell and activates p38 is not a compound disclosed in the patents and patent applications listed in Table 1.

In another aspect, the invention relates to a pharmaceutical composition, comprising a pharmaceutically acceptable carrier and a compound that increases the oxidative stress of a cell and activates p38, or a pharmaceutical salt of such a compound. In one embodiment, the pharmaceutical composition can include one or more additional therapeutic agent. In another embodiment, the pharmaceutical composition can include one or more additional chemotherapeutic agent. In one embodiment, the pharmaceutical composition does not include a compound disclosed in the patents and patent applications listed in Table 1.

In another aspect, the compound that increases oxidative stress in a cell induces the production of Hsp70 in the cell. In one embodiment, the compound that increases oxidative stress in a cell induces the production of Hsp70 to a greater extent in cancer cells compared to normal cells. For example, the compound induces the production of Hsp70 2-fold, 5-fold, 10-fold, 100-fold or more in cancer cells compared to non-cancerous cells. In some embodiments, the compound that induces the production of Hsp70 increases the amount of membrane-bound Hsp70 in cancer cells after the administration of the compound that induces the production of Hsp70 compared to the membrane-bound Hsp70 in the cancer cells before administration. In some embodiments, the compound that induces the production of Hsp70 increases the amount of Hsp70 secreted by the cancer cells after the administration of the compound that induces the production of Hsp70 compared to the Hsp70 secreted by the cancer cells before administration.

Heat shock proteins (HSPs) are found in virtually all prokaryotic and eukaryotic cells where they support folding of nascent polypeptides, prevent protein aggregation, and assist transport of other proteins across membranes. The proteins in the Hsp70 family (referred to collectively as “Hsp70”) play a dual role of protecting cells from lethal damage after environmental stress, on the one hand, and targeting cells for immune mediated cytolytic attack on the other hand. Increased expression of Hsp70 in the cytoplasma is known to protect a broad range of cells under stress (e.g., oxidative stress, heat stress, toxins such as heavy metals, radiation; excess/lack of nutrients or metabolic products) by preventing the misfolding, aggregation and denaturation of cytoplasmic proteins (Mosser, et al., Mol Cell Biol. 2000 October; 20(19): 7146-7159; Yenari, Adv Exp Med Biol, 2002, 513, 281-299; Kiang and Tsokos, Pharmacol Ther. 1998; 80(2):182-201). However, if cells are unable to compensate for a prolonged or excessive environmental stress, they secrete Hsp70 and display membrane-bound Hsp70 which flags them for cytolytic attack mediated by natural killer cells.

Heat shock proteins are regulated by the transcription factor, heat shock factor-1 (HSF-1), which binds to heat shock elements (HSE) which are found in all heat shock genes. Under normal growing conditions, HSF-1 exists in a latent monomeric form in the cytoplasm. In response to heat shock or other stresses, HSF-1 is translocated to the nucleus where it trimerizes and binds to HSE. The activation of HSF-1 by oxidative stress has been reported in the literature to require p38 kinase signaling in some cell lines.

Natural killer (NK) cells, a type of white blood cell, are known to be an important component of the body's immune system. Because the defining function of NK cells is spontaneous cytotoxicity without prior immunization, NK cells can be the first line of defense in the immune system, and are believed to play a role in attacking cancer cells and infectious diseases. It is believed that Hsp70 secreted by a cell acts as a chemoattractant for NK cells and that membrane-bound Hsp70 provides a target structure that is recognized by CD94 receptors on the surface of NK cells and causes them to produce cytotoxic granules that contain high amounts of granzyme B which induces apoptosis in the target cell. Cytotoxic granules are thought to enter the target cell with the assistance of perforin, a membrane disrupting protein produced by NK cells (see Radons and Multhoff, Exerc. Immunol. Rev. (2005), 11:17-33) and Loeb, et al., Journal of Biological Chemistry (2006), www.jbc.org/cgi/doi/10.1074/jbc.M604544200).

Hsp70 and NK cells have been shown to play a crucial role in tumor surveillance. Membrane-bound and soluble Hsp70 are involved in the activation of an anti-tumor immune response that is mediated by NK cells. Interestingly, the highly successful anti-cancer drug, paclitaxel, has been shown to exploit this immune response by increasing the secretion of perforin by NK cells which presumably increase their ability to deliver the apoptosis inducing protein, granzyme B, to the target tumor cell (Kubo, et al., Cancer Immunol. Immunother. (2005), 54:468-476). The enormous success of paclitaxel illustrates the need for new anti-cancer agents that augment the body's tumor immunesurveillence systems.

### BRIEF DESCRIPTION OF THE DRAWINGS

FIG. 1 is the structure of Taxol® (paclitaxel).

FIG. 2 is the structure of Taxotere® (docetaxel).

FIGS. 3-23 each depict the structure of particular Taxol® analogs.

FIG. 24 is the structure of a polymer comprising a Taxol® analog group pendent from the polymer backbone. The polymer is a terpolymer of the three monomer units shown.

FIG. 25 is a photograph of a gel showing that Compound 1 preferentially induces Hsp70 in MDA-MB-435 tumor cells compared to non-tumor HMEC and HRE cells.

FIG. 26 is a graph showing the percent lysis of target cells that had been treated with Compound 1 (blue bars) compared to cells which were treated with vehicle alone (yellow bars). Treatment with Compound 1 increased lysis of MDA-MB-435 tumor cell line by about 15% compared to MDA-MB-435 cells which were treated with DMSO alone. Treatment with Compound 1 only slightly increased the lysis of non-cancerous HMEC and HRE cells compared to cells treated with DMSO alone.

FIG. 27A is an image captured with a monochrome CCD camera SenSys showing that MCF-7 cells which have not been treated with Compound 1 do not have detectable Hsp70 on the surface of cells. Hsp70 is indicated by a bright pink fluorescent color.

FIG. 27B is an image captured with a monochrome CCD camera SenSys showing that MCF-7 cells which have not been treated with the primary antibody which binds to Hsp70 do not have a bright pink color fluorescence.

FIG. 27C is an image captured with a monochrome CCD camera SenSys showing that MCF-7 cells which have been treated with 0.5 μM of Compound 1 for 3 hrs. have a bright pink fluorescent color on the surface of the cells indicating Hsp70.

FIG. 27D is an image captured with a monochrome CCD camera SenSys showing that MCF-7 cells which have been treated with 0.5 μM of Compound 1 for 3 hrs. then have been allowed to recover for 2 hrs. in the absence of Compound 1 have a bright pink fluorescent color on the surface of the cells indicating Hsp70 which is stronger than immediately following treatment with Compound 1.

FIG. 28A is an image captured with a monochrome CCD camera SenSys showing that CEM cells which have not been treated with Compound 1 do not have detectable Hsp70 on the surface of cells. Hsp70 is indicated by a bright pink fluorescent color.

FIG. 28B is an image captured with a monochrome CCD camera SenSys showing that CEM cells which have been treated with 0.05 μM of Compound 1 for 3 hrs. have a bright pink fluorescent color on the surface of the cells indicating Hsp70.

FIG. 28C is an image captured with a monochrome CCD camera SenSys showing that CEM cells which have been treated with 0.1 μM of Compound 1 for 3 hrs. have a bright pink fluorescent color on the surface of the cells indicating Hsp70. The fluorescent color indicating Hsp70 is brighter than when cells were treated with 0.05 μM of Compound 1 indicating more Hsp70 on the surface of the cell.

FIG. 28D is an image captured with a monochrome CCD camera SenSys showing that CEM cells which have been treated with 0.5 μM of Compound 1 for 3 hrs. have a bright pink fluorescent color on the surface of the cells indicating Hsp70. The fluorescent color indicating Hsp70 is brighter than when cells were treated with 0.05 μM of Compound 1 but about the same as cells treated with 0.1 KIM indicating that induction of Hsp70 on the surface of the cells may plateau at concentrations greater than 0.1 μM.

FIG. 29A and FIG. 29 B are images captured with by confocal microscopy showing that HT29 cells which have not been treated with Compound 1 do not have detectable Hsp70 on the surface of cells. Hsp70 is indicated by a bright green fluorescent color.

FIG. 29C and FIG. 29 D are images captured with by confocal microscopy showing that HT29 cells which have been treated with 500 nM of Compound 1 for 6 hrs have a bright green color on the surface of the cells indicating Hsp70.

FIG. 29E is a Z-section of an HT29 cell which has been treated with Compound 1.

FIG. 30 is a graph showing the amount of Hsp70 secreted by MDA-MB-435 tumor cells treated with Compound 1 or untreated indicating that Compound 1 increased the secretion of Hsp70 by the cells in a dose dependent manner.

FIG. 31 is a graph showing cell survival as a percentage of control in cells treated with DMSO alone (control), in cells treated with N-acetyl-L-cysteine (NAC) an antioxidant, and in cells treated with 500 nM of Compound 1 in the presence of increasing concentrations of NAC (0 mM, 0.1 mM, 0.5 mM, 1.0 mM, or 2.5 mM). 2.5 mM of NAC completely abolished the ability of Compound 1 to induce apoptosis.

FIG. 32 is a graph showing the percentage of early stage apoptotic Ramos cells when the cells are treated with DMSO (control), NAC (5 mM), Compound 1 (0.01 μM, 0.05 μM, or 0.2 μM) and NAC (5 mM) plus Compound 1 (0.01 μM, 0.05 μM, or 0.2 μM). As can be seen by the graph, treatment with Compound 1 in combination with 5 mM of the antioxidant NAC completely blocks apoptosis by Compound 1 in Ramos cells.

FIG. 33 is a diagram showing the percentage of early stage apoptotic HSB2 cells when the cells are treated with (A) DMSO (control), (B) NAC (5 mM), (C) Compound 1 (0.2 μM) or (D) NAC (5 mM) plus Compound 1 (0.2 μM). Only 12% and 11% of the cells were in the early stage of apoptosis when treated with DMSO (FIG. 33A) or NAC (FIG. 33B), respectively. When cells were treated with 0.2 mM of Compound 1, 45% of the cells were in the early stages of apoptosis (FIG. 33C). Treatment with Compound 1 in combination with the antioxidant NAC (FIG. 33D) completely blocked apoptosis by Compound 1 and only 6% of the cells were in the early stages of apoptosis.

FIG. 34 is a graph showing two quantitative PCR experiments to measure the induction of Hsp70 by Compound 1 with and without the presence of the antioxidant NAC. Melanoma Hs294 T cells were treated with either DMSO (control), NAC (1 nM, 5 mM, or 10 mM) alone, Compound 1 (100 nM) alone, or Compound 1 (100 nM) in combination with NAC (1 mM, 5 mM or 10 mM). As can be seen from FIG. 34, 5 mM of NAC blocked the induction of Hsp70 by Compound 1.

FIG. 35 is a Western blot showing that Compound 1 induces phosphorylation and activation of p38 in a dose dependent manner.

FIG. 36 is a Western blot showing that Compound 1 induces the phosphorylation of MSK1, a downstream target of p38, in a dose dependent manner.

FIG. 37 is a Western blot showing that the phosphorylation of MSK1 induced by Compound 1 can be blocked by the specific p38 inhibitor SB203580.

FIG. 38 is a Western blot showing that Compound 1 induces the phosphorylation of MAPKAPK-2, a downstream target of p38, in CEM and CV-1 tumor cells and that this phosphorylation can be blocked by the specific p38 inhibitor, SB203580.

FIG. 39 is a diagram showing the percentage of early and late stage apoptotic HSB2 cells when the cells are treated with (A) DMSO (control), (B) NAC (5 mM), (C) an inhibitor of p38 (20 μM), (D) Compound 1 (0.2 μM), (E) NAC (5 mM) plus Compound 1 (0.2 μM) or (F) an inhibitor of p38 plus Compound 1. Only a small percentage of the cells are in the early and late stage of apoptosis when the cells were treated with DMSO (FIG. 39A), NAC (FIG. 39B), or the p38 inhibitor SB203580 (FIG. 39C). When cells are treated with 0.2 mM of Compound 1 26.6% of the cells were in the early stages of apoptosis and 41.5% of cells were in late stage apoptosis (FIG. 39D). Treatment with Compound 1 in combination with the antioxidant NAC (FIG. 39DE) completely blocked apoptosis by Compound 1 and only 2.9% of the cells are in the early stages of apoptosis and 3.8% of cells were in the late stages of apoptosis. In contrast, treatment of cells with Compound 1 in combination with the p38 inhibitor (FIG. 39DF) did not effect induction of apoptosis by Compound 1.

FIG. 40 is a graph showing that induction of Hsp70 by Compound 1 in Hs294 T cells was blocked by a p38 inhibitor and by NAC.

FIG. 41 is a graph showing that Ramos cells treated with Compound 1 emit more reactive oxygen species than cells treated with DMSO alone. The production of reactive oxygen species by the cells was measured using a cell permeable DCF-DA probe which, when oxidized by reactive oxygen species, emits a green fluorescence that was detected by flow cytometry.

FIG. 42 is a graph showing that induction of reactive oxygen species by Compound 1 can be blocked by treating Ramos cells with NAC in combination with Compound 1. The production of reactive oxygen species was measured using a DCF-DA probe and detected using flow cytometry.

FIG. 43 is a graph showing that Compound 1 induced the production of reactive oxygen species in Ramos cells in a time dependent manner. The production of reactive oxygen species was measured using a DCF-DA probe and detected using flow cytometry.

FIG. 44 is a graph showing both Tiron and NAC block the production of reactive oxygen species induced by treatment of cells with Compound 1. Tiron and NAC are antioxidants that function by different mechanisms. The production of reactive oxygen species was measured using a DCF-DA probe and detected using flow cytometry.

FIG. 45 is a graph showing Tiron blocks the induction of Hsp70 by Compound 1.

FIG. 46 is a graph showing the average tumor volume in milliliters over time (in days) in nude mice treated with vehicle; Compound (1) (50 mg/kg); Paclitaxel (5 mg/kg); Compound (1) (25 mg/kg) plus Paclitaxel (5 mg/kg); or Compound (1) (50 mg/kg) plus Paclitaxel (5 mg/kg). The tumors were generated from the human breast tumor cell line MDA-435.

FIG. 47 is a graph showing the percent weight change over time in nude mice treated with vehicle; Compound (1) (50 mg/kg); Paclitaxel (5 mg/kg); Compound (1) (25 mg/kg) plus Paclitaxel (5 mg/kg); or Compound (1) (50 mg/kg) plus Paclitaxel (5 mg/kg). The mice were being treated for tumors generated from the human breast tumor cell line MDA-435.

FIGS. 48A, 48B, and 48C are bar graphs showing the percent increase in Hsp70 plasma levels associated with administration of the Compound (1)/paclitaxel combination therapy at 1 hour (FIG. 48A), 5 hours (FIG. 48B), and 8 hours (FIG. 48C) after administration.

FIG. 49 is a graph showing percent of elesclomol-treated Ramos cells with DCFDA fluorescence relative to DMSO-treated control Ramos cells over time after treatments.

FIG. 50 is a graph showing averaged fold of Hsp70 RNA induction in Ramos cells treated with elesclomol relative to that in Ramos cells treated with DMSO (control).

FIG. 51(a) show Western blots probed with Cytochrome C primary antibody, showing activation of cytochrome c in cytosol and mitochontria of Ramos cells treated with elesclomol and DMSO (control) over time.

FIG. 51(b) show Western blots probed with Caspase 3 antibody and cleaved Caspase 3 primary antibody, showing activation of caspase 3 in Ramos cells treated with elesclomol and DMSO (control) over time.

### DETAILED DESCRIPTION OF THE INVENTION

### I. Definition of Terms

As used herein, the phrase “a compound that increases oxidative stress in a cell” refers to a compound that increases the production of intracellular reactive oxygen species (e.g., superoxide anion, hydrogen peroxide, hydroxyl radical), increasing the expression or activity of enzymes which generate reactive oxygen species (e.g., NADPH oxidase and dual oxidases), and/or depleting or inactivating protective reducing metabolites or enzymes (e.g., superoxide dismutase (SOD) isoenzymes (CuZnSOD, MnSOD and ecSOD), glutathione, glutathione peroxidase, catalase, nicotinamide adenine dinucleotide phosphate (NADPH), thioredoxin, thioredoxin reductase). The phrase “a compound of the invention” refers to a compound that increases the oxidative stress of a cell and increases the activity of p38. When a compound that increases the oxidative stress of a cell is administered to a subject in combination with another anti-cancer therapy, the compound increases the oxidative stress of the cancer cells more than the anti-cancer therapy alone would increase the oxidative stress. Compounds that can increase oxidative stress in a cell and increase the activity of p38 include, but are not limited to, nucleic acids, antisense nucleic acids, ribozyme, triple helix, antibody, antibody fragments, peptide molecules, cytokines, cytokine fragments, and small inorganic or organic molecules (preferably, small organic molecules having a molecule weight of about 1000 or less, more preferably the molecular weight is 500 or less). Compounds that increase oxidative stress and p38 activity in a cell are disclosed in the patents and patent applications listed in Table 1, the entire teachings of which are incorporated herein by reference.

As used herein, “Hsp70” includes each member of the family of heat shock proteins having a mass of about 70-kiloDaltons, including forms such as constituitive, cognate, cell-specific, glucose-regulated, inducible, etc. Examples of specific Hsp70 proteins include hsp70, hsp70hom; hsc70; Grp78/BiP; mt-hsp70/Grp75, and the like). Typically, the disclosed methods increase expression of inducible Hsp70. Functionally, the 70-kDa HSP (HSP70) family is a group of chaperones that assist in the folding, transport, and assembly of proteins in the cytoplasm, mitochondria, and endoplasmic reticulum. Membrane-bound Hsp70, however, provides a target structure for cytolytic attack mediated by natural killer cells. In humans, the Hsp70 family encompasses at least 11 genes encoding a group of highly related proteins. See, for example, Tavaria, et al., Cell Stress Chaperones, 1996; 1(1):23-28; Todryk, et al., Immunology. 2003, 110(1): 1-9; and Georgopoulos and Welch, Annu Rev Cell Biol. 1993; 9:601-634; the entire teachings of these documents are incorporated herein by reference.

The phrase “increases cellular expression of Hsp70,” as used herein, refers to increasing the production of Hsp70 in a cell at the transcriptional or translational level, or increases an activity of Hsp70. Increased cellular expression of Hsp70 can be measure by any method known in the art or by the method described in Example 1 herein. In one embodiment, a compound that increases cellular expression of Hsp70 increases the amount of membrane-bound Hsp70 in a cell by, for example, increasing the production of Hsp70, by translocating Hsp70 to the membrane of the cell, or by a combination of increasing the production of Hsp70 in the cell and translocating Hsp70 to the membrane of the cell. Membrane bound Hsp70 can be measured by any method known in the art or by the method described in Example 4. In another embodiment, a compound that increases cellular expression of Hsp70 increases the amount of Hsp70 secreted by a cell by, for example, increasing the production of Hsp70, by translocating Hsp70 to the membrane of the cell, or by a combination of increasing the production of Hsp70 in the cell and translocating Hsp70 to the membrane of the cell. Hsp70 secretion by a cell may be measured by any method known in the art or by the method described in Example 5. A significant increase in the expression of Hsp70, membrane bound Hsp70 or secreted Hsp70 can be indicated by an increase of 20%, 30%, 40%, 50%, 60%, 70%, 80%, 90%, 100%, 200% or more. Compounds that increase cellular expression of Hsp70 include, but are not limited to, nucleic acids, antisense nucleic acids, ribozyme, triple helix, antibody, antibody fragments, peptide molecules, cytokines, cytokine fragments, and small inorganic or organic molecules (preferably, small organic molecules having a molecule weight of about 1000 or less, more preferably, small organic molecules have a molecular weight of 500 or less). Compounds that increase cellular expression of Hsp70 are disclosed in the patents and patent applications listed in Table 1, the entire teachings of which are incorporated herein by reference.

TABLE 1

| United States Issued Patents | |
|---|---|
| Pat. No. | Issue Date |
| 6,762,204 | Jul. 13, 2004 |
| 6,800,660 | Oct. 5, 2004 |
| 6,825,235 | Nov. 30, 2004 |
| 6,924,312 | Aug. 2, 2005 |
| 7,001,923 | Feb. 21, 2006 |
| 7,037,940 | May 2, 2006 |
| 7,074,952 | Jul. 11, 2006 |
| United States Applications | |
| application No. | Filing Date |
| 10/307,916 | Dec. 2, 2002 |
| 10/758,589 | Jan. 15, 2004 |
| 11/157,213 | Jun. 20, 2005 |
| 11/244,324 | Oct. 5, 2005 |
| 11/244,427 | Oct. 5, 2005 |
| 11/432,307 | May 11, 2006 |
| 11/440,429 | May 24, 2006 |
| United States Provisional | |
| Applications | |
| Provisional Ser. No. | Filing Date |
| 60/839,034 | Aug. 21, 2006 |
| 60/839,042 | Aug. 21, 2006 |
| 60/839,064 | Aug. 21, 2006 |
| 60/839,066 | Aug. 21, 2006 |
| 60/841,408 | Aug. 31, 2006 |

Mitogen-activated protein kinases (MAP kinases) are a family of proline-directed serine/threonine kinases that activate their substrates by dual phosphorylation. The kinases are activated by a variety of signals including oxidative stress, nutritional and osmotic stress, UV light, growth factors, endotoxin and inflammatory cytokines. The p38 MAP kinase group is a MAP family of various isoforms, including p38α, p38β and p38γ, and is responsible for phosphorylating and activating transcription factors (e.g. ATF2, CHOP and MEF2C) as well as other kinases (e.g. MSK1, MAPKAPK-2). The p38 isoforms are activated by oxidative stress, bacterial lipopolysaccharide, physical and chemical stress and by pro-inflammatory cytokines, including tumor necrosis factor (TNFα) and interleukin-1 (IL-1). The products of the p38 phosphorylation mediate the production of inflammatory cytokines, including TNF and IL-1, and cyclooxygenase-2.

Increase in the activity of p38 kinase in cells that have been contacted with an agent can be measured by any method known to those skilled in the art. In one embodiment, increase in the activity of p38 kinase in a group of cells can be measured by determining the phosphorylation state of p38 kinase in a subset of the cells that have not been contacted with the agent and in a subset of the cells that have been contacted with the agent, wherein increased phosphorylation of p38 kinase in the subset of cells contacted with the agent compared to the subset of cells not contacted with the agent indicates that the agent increases the activity of p38 kinase. A significant increase in the activity of p38 kinase can be indicated by an increase of 20%, 30%, 40%, 50%, 60%, 70%, 80%, 90%, 100%, 200% or more in the phosphorylation state of p38 kinase. This method is further described in Example 8. Increase in p38 activity can also be measured by measuring the phosphorylation state of downstream targets of p38 such as MSK1 and MAPKAPK-2, wherein an increase in phosphorylation of MSK1 and MAPKAPK-2 indicates an in p38 activity. Methods for measuring increased phosphorylation of MSK1 and MAPKAPK-2 are described in Examples 10 and 11, respectively.

Mitogen- and stress-activated protein kinase 1 (MSK1) is activated on stimulation of the Ras-mitogen activated protein kinase (MAP kinase) pathway, where it can be activated by phosphorylation by p38 kinase.

Mitogen-activated protein kinase-activated protein kinase 2(MAPKAPK-2) is activated on stimulation of the p38 kinase pathway, where it is activated by phosphorylation by p38 kinase.

The term “natural killer (NK) cells,” as used herein, is a type of lymphocyte necessary for mammalian defense against virus infections and transformed cells. It is believed that membrane-bound Hsp70 or secreted Hsp70 from cells provides a target structure that is recognized by CD94 receptors on the surface of NK cells and causes them to produce cytotoxic granules that contain high amounts of granzyme B and perforin which induces apoptosis in the target cell. (see Radons and Multhoff, Exerc. Immunol. Rev. (2005), 11:17-33). Perforin is a protein that disrupts the membrane of the target cell, whereas granzymes are a family of structurally related serin proteases with various substrates specificities. Granzyme B has been shown to cleave target-cell proteins at specific aspartate residues and is the most potent activator of caspase-mediated, as well as caspase-independent apoptosis.

A compound that sensitizes cells for natural killer cell induced cytotoxicity is an compound that increases the likelihood that NK cells will recognize and target the cell for cytotoxic lysis. For example, membrane-bound Hsp70 and Hsp70 secreted by a target cell can bind to CD94 receptors of NK cells and cause them to release cytotoxic granules containing perforin and granzyme B which induce cytotoxicity in the target cell. In addition, Hsp70 secreted by a target cell forms a microenvironment around the target cell which has a higher concentration of Hsp70 and acts as a chemoattractant for NK cells.

As used herein, the term “pharmaceutically acceptable salt,” is a salt formed from, for example, an acid and a basic group of one of a compound of the invention. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of the invention having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The term “pharmaceutically acceptable salt” also refers to a salt prepared from a compound of the invention having a basic functional group, such as an amine functional group, and a pharmaceutically acceptable inorganic or organic acid. Suitable acids include, but are not limited to, hydrogen sulfate, citric acid, acetic acid, oxalic acid, hydrochloric acid (HCl), hydrogen bromide (HBr), hydrogen iodide (HI), nitric acid, hydrogen bisulfide, phosphoric acid, lactic acid, salicylic acid, tartaric acid, bitartratic acid, ascorbic acid, succinic acid, maleic acid, besylic acid, fumaric acid, gluconic acid, glucaronic acid, formic acid, benzoic acid, glutamic acid, methanesulfonic acid, ethanesulfonic acid, benzenesulfonic acid, and p-toluenesulfonic acid.

As used herein, the term “anti-cancer therapy,” refers to chemotherapy, radiotherapy (including, but not limited to, gamma-radiation, neutron beam radiotherapy, electron beam radiotherapy, proton therapy, brachytherapy, and systemic radioactive isotopes), endocrine therapy, biologic response modifiers (including, but not limited to, interferons, interleukins, and tumor necrosis factor (TNF)), hyperthermia and cryotherapy. A preferred anti-cancer therapy that can be used in combination with a compound that increases the oxidative stress of cancer cells is chemotherapy.

Chemotherapeutic agents that can be used in combination with a compound that increases the oxidative stress and p38 activity in a cell include, for example, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other chemotherapeutic agents that can be used in combination with a compound that increases the oxidative stress and p38 activity in a cell include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Chemotherapeutic agents that can be used in combination with a compound that increases the oxidative stress and p38 activity in a cell include therapeutic antibodies include such as HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Chemotherapeutic agents that can be used in combination with a compound that increases the oxidative stress and p38 activity in a cell include, but are not limited to, alkylating agents, antimetabolites, natural products, or hormones. Examples of alkylating agents useful in the methods of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful in the methods of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful in the methods of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and with the compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Chemotherapeutic agents that can be used in combination with a compound that increases the oxidative stress and p38 activity in a cell include, but are not limited to, microtubulin inhibitors. As used herein, a “microtubulin inhibitor” means an anti-cancer agent which acts by inhibiting tubulin polymerization or microtubule assembly. Examples of microtubulin inhibitors include without limitation the following marketed drugs and drugs in development: Erbulozole (also known as R-55104); Dolastatin 10 (also known as DLS-10 and NSC-376128); Mivobulin isethionate (also known as CI-980); Vincristine; NSC-639829; ABT-751 (Abbot, also known as E-7010); Altorhyrtins (such as Altorhyrtin A and Altorhyrtin C); Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9); Cemadotin hydrochloride (also known as LU-103793 and NSC-D-669356); Auristatin PE (also known as NSC-654663); Soblidotin (also known as TZT-1027); LS-4559-P (Pharmacia, also known as LS-4577); LS-4578 (Pharmacia, also known as LS-477-P); LS-4477 (Pharmacia), LS-4559 (Pharmacia); RPR-112378 (Aventis); Vincristine sulfate; DZ-3358 (Daiichi); GS-164 (Takeda); GS-198 (Takeda); KAR-2 (Hungarian Academy of Sciences); SAH-49960 (Lilly/Novartis); SDZ-268970 (Lilly/Novartis); AM-97 (Armad/Kyowa Hakko); AM-132 (Armad); AM-138 (Armad/Kyowa Hakko); IDN-5005 (Indena); Cryptophycin 52 (also known as LY-355703); Vitilevuamide; Tubulysin A; Canadensol; Centaureidin (also known as NSC-106969); T-138067 (Tularik, also known as T-67, TL-138067 and TI-138067); COBRA-1 (Parker Hughes Institute, also known as DDE-261 and WHI-261); H10 (Kansas State University); H16 (Kansas State University); Oncocidin A1 (also known as BTO-956 and DIME); DDE-313 (Parker Hughes Institute); SPA-2 (Parker Hughes Institute); SPA-1 (Parker Hughes Institute, also known as SPIKET-P); 3-IAABU (Cytoskeleton/Mt. Sinai School of Medicine, also known as MF-569); Narcosine (also known as NSC-5366); Nascapine, D-24851 (Asta Medica), A-105972 (Abbott); Hemiasterlin; 3-BAABU (Cytoskeleton/Mt. Sinai School of Medicine, also known as MF-191); TMPN (Arizona State University); Vanadocene acetylacetonate; T-138026 (Tularik); Monsatrol; Inanocine (also known as NSC-698666); 3-IAABE (Cytoskeleton/Mt. Sinai School of Medicine); A-204197 (Abbott); T-607 (Tularik, also known as T-900607); RPR-115781 (Aventis); Eleutherobins (such as Desmethyleleutherobin, Desaetyleleutherobin, Isoeleutherobin A, and Z-Eleutherobin); Halichondrin B; D-64131 (Asta Medica); D-68144 (Asta Medica); Diazonamide A; A-293620 (Abbott); NPI-2350 (Nereus); TUB-245 (Aventis); A-259754 (Abbott); Diozostatin; (−)-Phenylahistin (also known as NSCL-96F037); D-68838 (Asta Medica); D-68836 (Asta Medica); Myoseverin B; D-43411 (Zentaris, also known as D-81862); A-289099 (Abbott); A-318315 (Abbott); HTI-286 (also known as SPA-110, trifluoroacetate salt) (Wyeth); D-82317 (Zentaris); D-82318 (Zentaris); SC-12983 (NCI); Resverastatin phosphate sodium; BPR-0Y-007 (National Health Research Institutes); SSR-250411 (Sanofi); Combretastatin A4; and analogs and derivatives thereof.

In a preferred embodiment, chemotherapeutic agents that can be used in combination with a compound that increases the oxidative stress and p38 activity in a cell include, but are not limited to, microtubulin stabilizers. As used herein, a “microtubulin stabilizer” means a chemotherapeutic agent which acts by arresting cells in the G2-M phases due to stabilization of microtubules. Examples of microtubulin stabilizers include paclitaxel and paclitaxel analogues. Additional examples of microtubulin stabilizers included without limitation the following marketed drugs and drugs in development: Discodermolide (also known as NVP-XX-A-296); Epothilones (such as Epothilone A, Epothilone B, Epothilone C (also known as desoxyepothilone A or dEpoA); Epothilone D (also referred to as KOS-862, dEpoB, and desoxyepothilone B); Epothilone E; Epothilone F; Epothilone B N-oxide; Epothilone A N-oxide; 16-aza-epothilone B; 21-aminoepothilone B (also known as BMS-310705); 21-hydroxyepothilone D (also known as Desoxyepothilone F and dEpoF), 26-fluoroepothilone); FR-182877 (Fujisawa, also known as WS-9885B), BSF-223651 (BASF, also known as ILX-651 and LU-223651); AC-7739 (Ajinomoto, also known as AVE-8063A and CS-39.HCl); AC-7700 (Ajinomoto, also known as AVE-8062, AVE-8062A, CS-39-L-Ser.HCl, and RPR-258062A); Fijianolide B; Laulimalide; Caribaeoside; Caribaeolin; Taccalonolide; Eleutherobin; Sarcodictyin; Laulimalide; Dictyostatin-1; Jatrophane esters; and analogs and derivatives thereof.

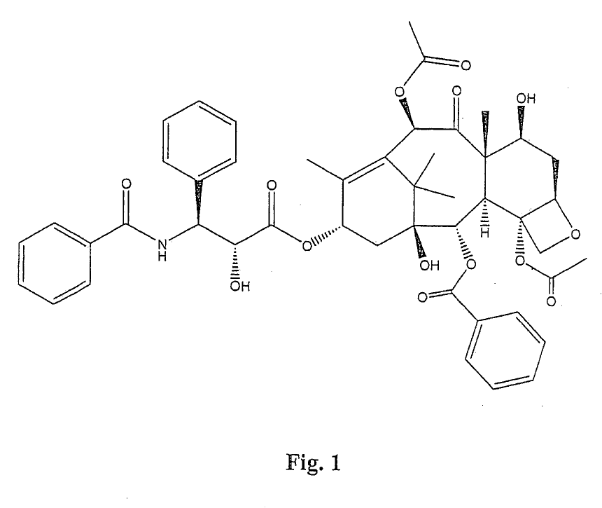

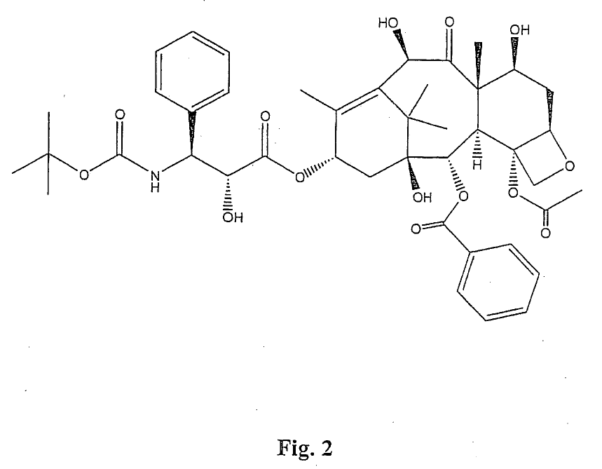

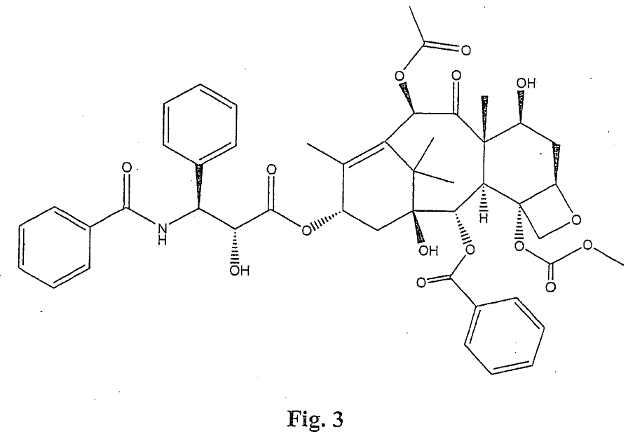

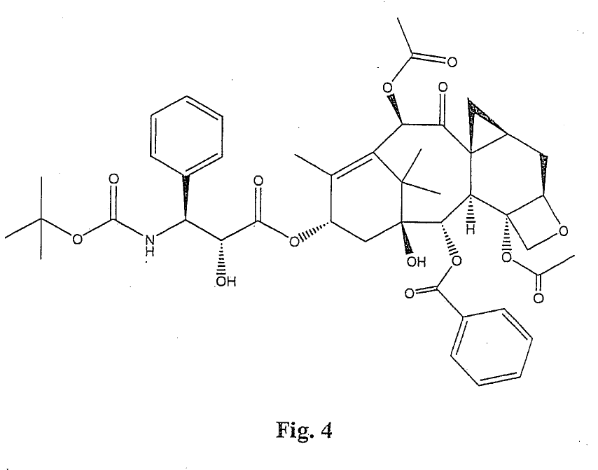

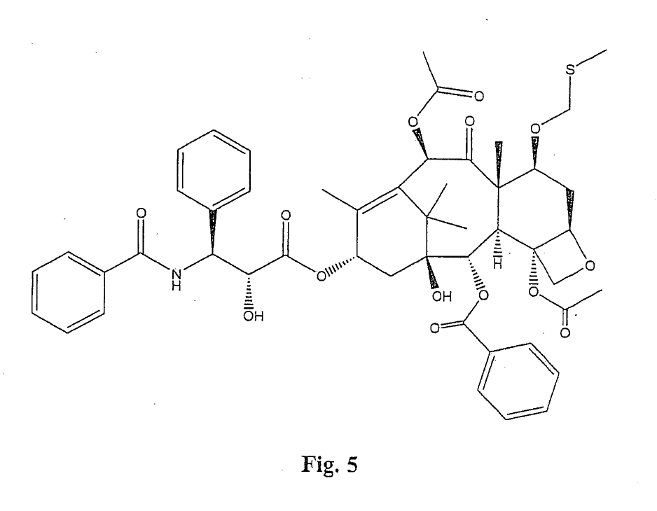

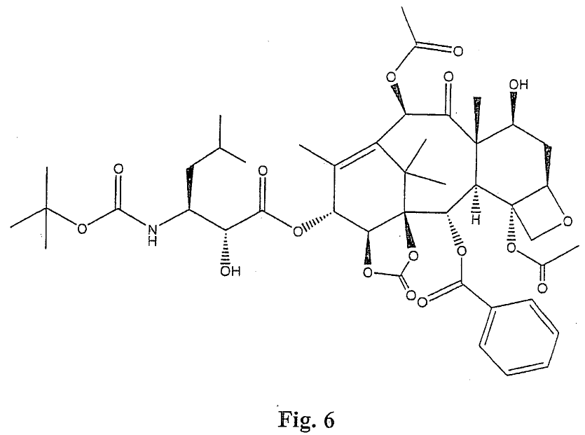

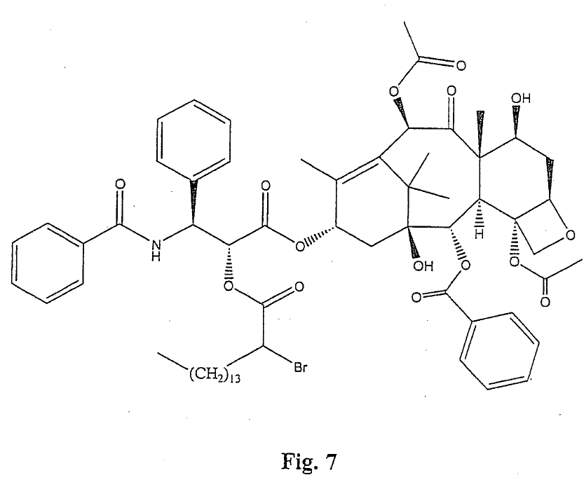

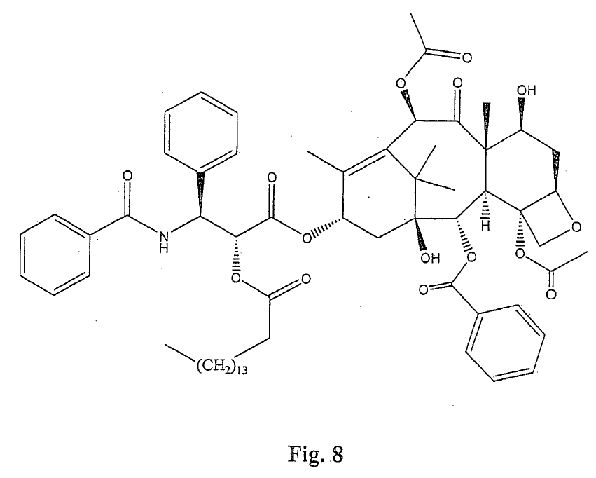

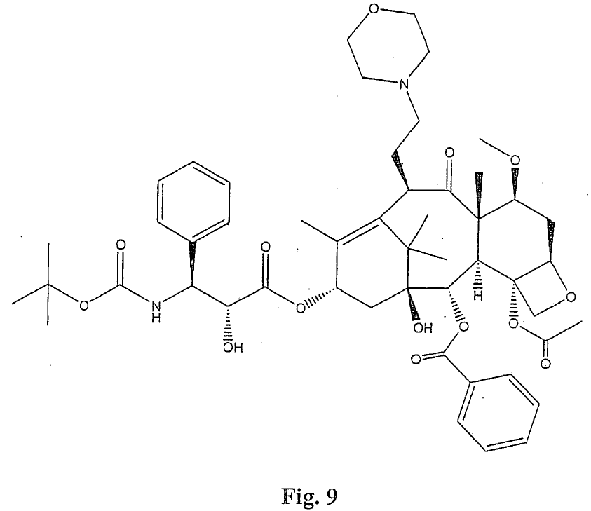

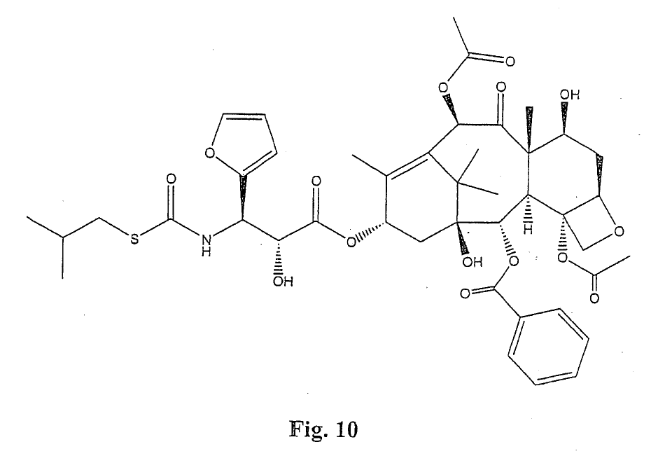

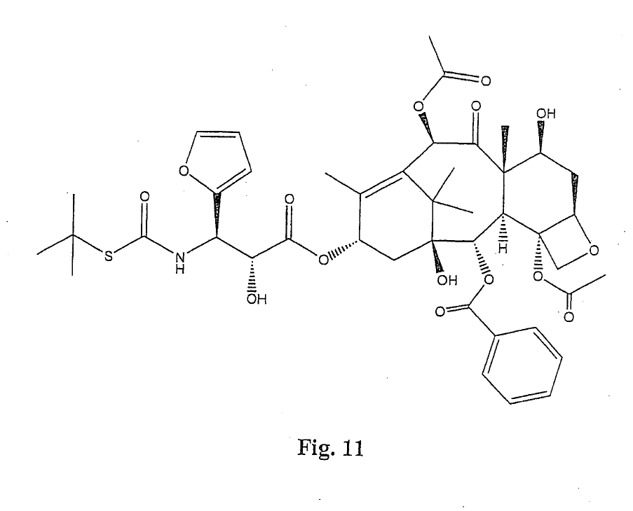

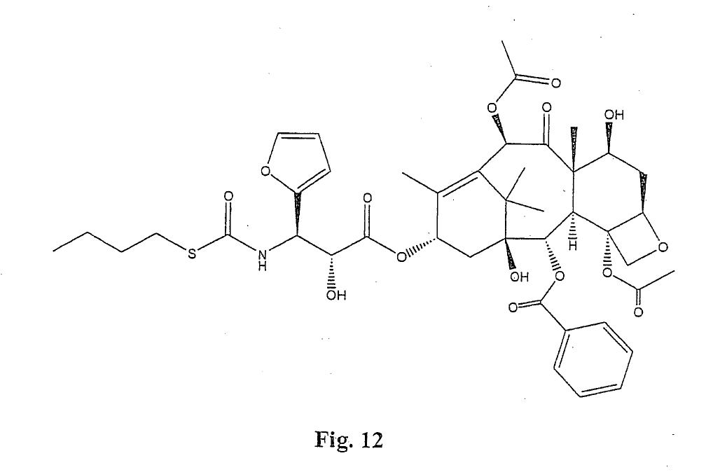

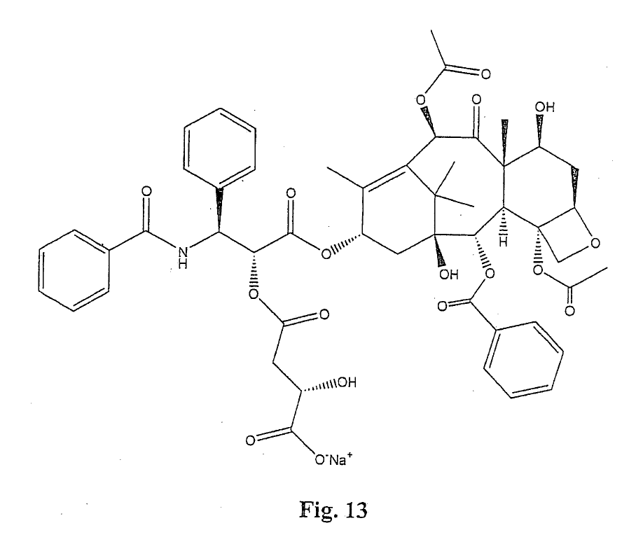

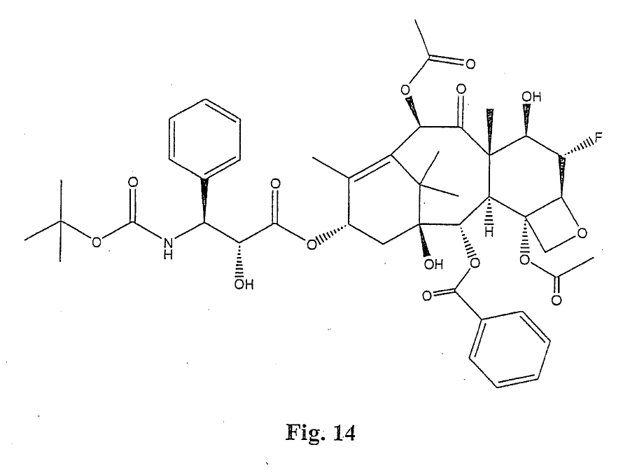

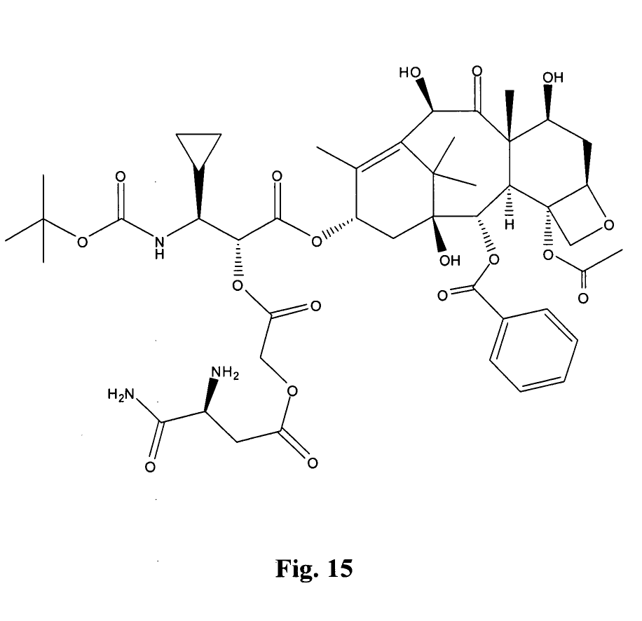

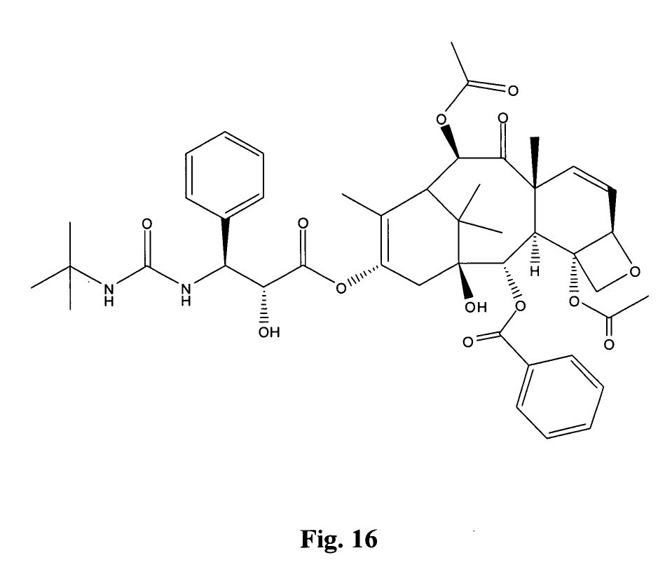

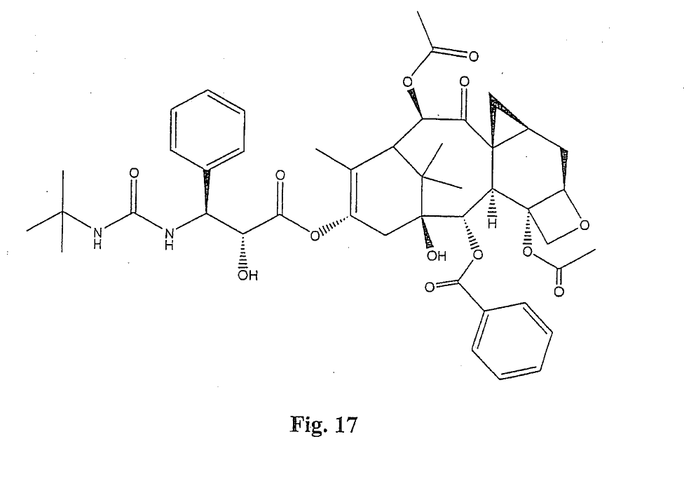

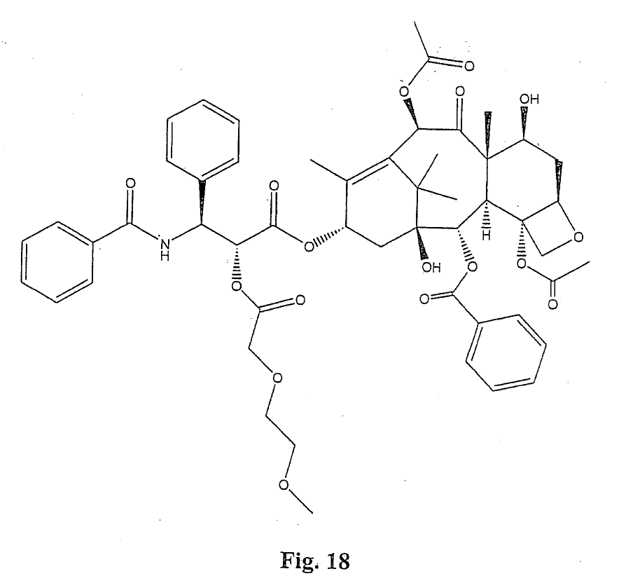

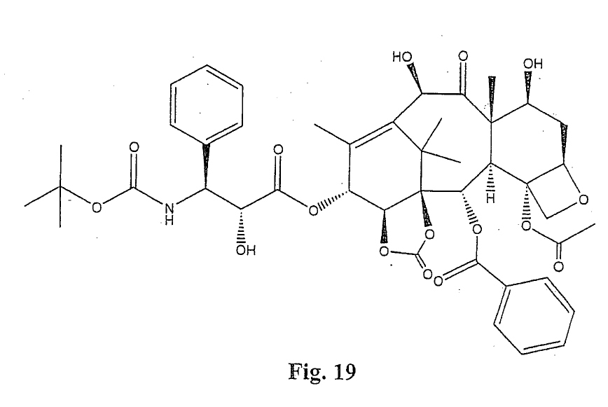

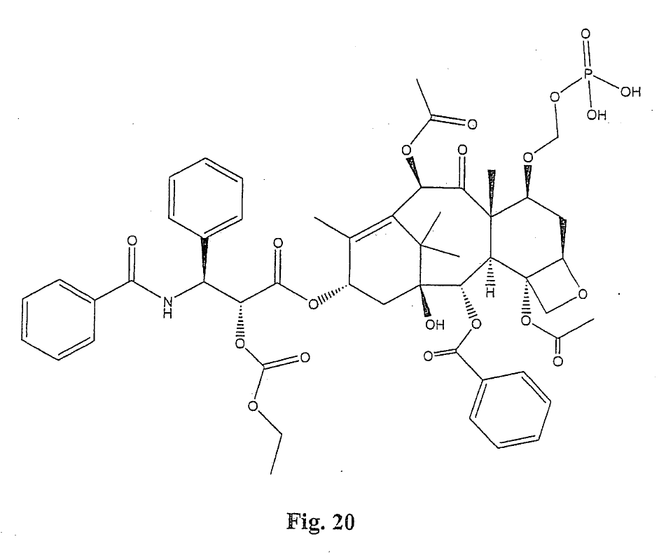

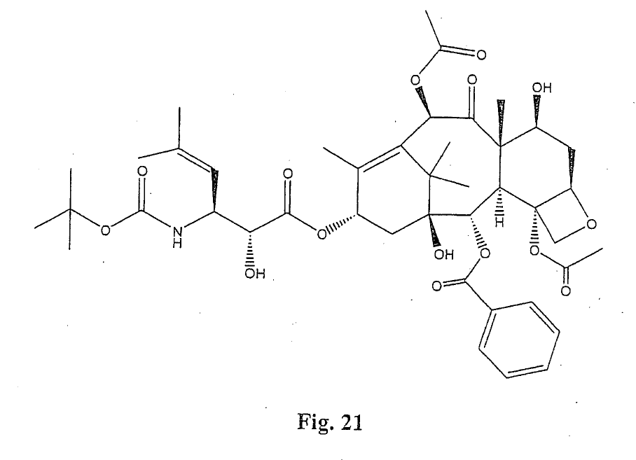

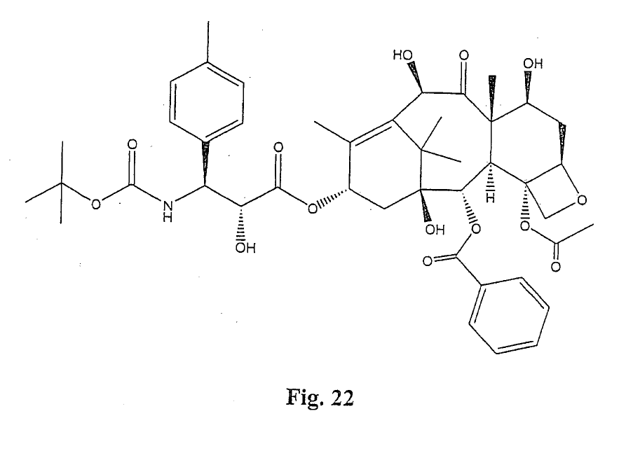

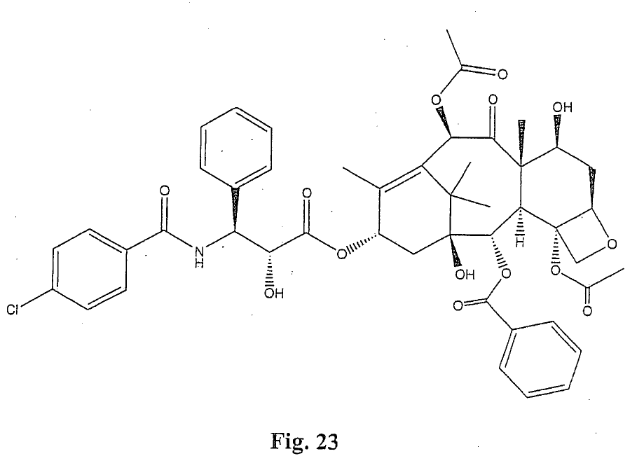

Paclitaxel and paclitaxel analogs are preferred chemotherapeutic agents that can be used in combination with a compound that increases the oxidative stress of a cell. Paclitaxel, also referred to as “Taxol®,” is a well-known chemotherapeutic agent which acts by enhancing and stabilizing microtubule formation. The structure of paclitaxel is shown in FIG. 1. Many analogs of paclitaxel are known, including docetaxel, the structure of which is shown in FIG. 2. Docetaxel is also referred to as “Taxotere®”. The structures of other paclitaxel analogs are shown in FIGS. 3-23. Paclitaxel analogs have the basic taxane skeleton as a common structure feature and have also been shown to have the ability to arrest cells in the G2-M phases due to stabilization of microtubules. Thus, it is apparent from FIGS. 3-23 that a wide variety of substituents can decorate the taxane skeleton without adversely affecting biological activity. It is also apparent that zero, one or both of the cyclohexane rings of a paclitaxel analog can have a double bond at the indicated positions. For clarity purposes, the basic taxane skeleton is shown below in Structural Formula (VI):

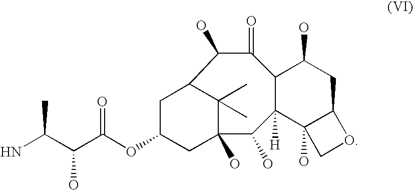

Double bonds have been omitted from the cyclohexane rings in the taxane skeleton represented by Structural Formula (VI). The basic taxane skeleton can include zero or one double bond in one or both cyclohexane rings, as indicated in FIGS. 3-23 and Structural Formulas (VII) and (VIII) below. A number of atoms have also been omitted from Structural Formula (VI) to indicate sites in which structural variation commonly occurs among paclitaxel analogs. For example, substitution on the taxane skeleton with simply an oxygen atom indicates that hydroxyl, acyl, alkoxy or another oxygen-bearing substituent is commonly found at the site. These and other substitutions on the taxane skeleton can be made without losing the ability to enhance and stabilize microtubule formation. Thus, the term “paclitaxel analog” is defined herein to mean a compound which has the basic taxane skeleton and which promotes microtubule formation. Paclitaxel analogs may be formulated as a nanoparticle colloidal composition to improve the infusion time and to eliminate the need to deliver the drug with Cremophor which causes hypersensitivity reactions in some patients. An example of a paclitaxel analog formulated as a nanoparticle colloidal composition is Abraxane which is a nanoparticle colloidal composition of protein-stabilized paclitaxel that is reconstituted in saline.

In some embodiments, the paclitaxel analogs used herein are represented by Structural Formula (VII) or (VIII):

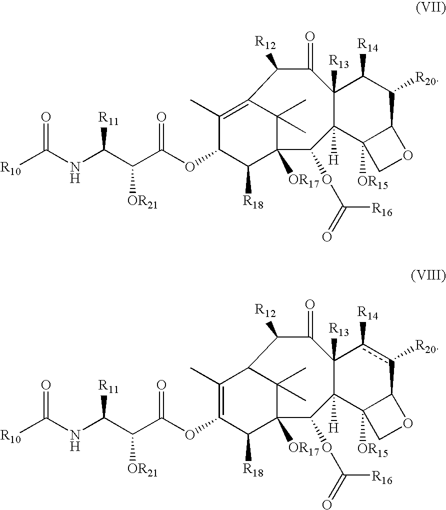

R₁₀ is a lower alkyl group, a substituted lower alkyl group, a phenyl group, a substituted phenyl group, —SR₁₉, —NHR₁₉ or —OR₁₉.

R₁₁ is a lower alkyl group, a substituted lower alkyl group, an aryl group or a substituted aryl group.

R₁₂ is —H, —OH, lower alkyl, substituted lower alkyl, lower alkoxy, substituted lower alkoxy, —O—C(O)-(lower alkyl), —O—C(O)-(substituted lower alkyl), —O—CH₂—O-(lower alkyl)-S—CH₂—O-(lower alkyl).

R₁₃ is —H, —CH₃, or, taken together with R₁₄, —CH₂—.

R₁₄ is —H, —OH, lower alkoxy, —O—C(O)-(lower alkyl), substituted lower alkoxy, —O—C(O)-(substituted lower alkyl), —O—CH₂—O—P(O)(OH)₂, —O—CH₂—O-(lower alkyl), —O—CH₂—S-(lower alkyl) or, taken together with R₂₀, a double bond.

R₁₅—H, lower acyl, lower alkyl, substituted lower alkyl, alkoxymethyl, alkthiomethyl, —OC(O)—O(lower alkyl), —OC(O)—O(substituted lower alkyl), —OC(O)—NH(lower alkyl) or —OC(O)—NH(substituted lower alkyl).

R₁₆ is phenyl or substituted phenyl.

R₁₇ is —H, lower acyl, substituted lower acyl, lower alkyl, substituted, lower alkyl, (lower alkoxy)methyl or (lower alkyl)thiomethyl.

R₁₈—H, —CH₃ or, taken together with R₁₇ and the carbon atoms to which R₁₇ and R₁₈ are bonded, a five or six membered a non-aromatic heterocyclic ring.

R₁₉ is a lower alkyl group, a substituted lower alkyl group, a phenyl group, a substituted phenyl group.

R₂₀ is —H or a halogen.

R₂₁ is —H, lower alkyl, substituted lower alkyl, lower acyl or substituted lower acyl.

Preferably, the variables in Structural Formulas (VII) and (VIII) are defined as follows: R₁₀ is phenyl, tert-butoxy, —S—CH₂—CH—(CH₃)₂, —S—CH(CH₃)₃, —S—(CH₂)₃CH₃, —O—CH(CH₃)₃, —NH—CH(CH₃)₃, —CH═C(CH₃)₂ or para-chlorophenyl; R₁₁ is phenyl, (CH₃)₂CHCH₂—, -2-furanyl, cyclopropyl or para-toluoyl; R₁₂ is —H, —OH, CH₃CO— or —(CH₂)₂—N-morpholino; R₁₃ is methyl, or, R₁₃ and R₁₄, taken together, are —CH₂—;

R₁₄ is —H, —CH₂SCH₃ or —CH₂—O—P(O)(OH)₂; R₁₅ is CH₃CO—;

R₁₆ is phenyl; R₁₇—H, or, R₁₇ and R₁₈, taken together, are —O—CO—O—;

R₁₈ is —H; R₂₀ is —H or —F; and R₂, is —H, —C(O)—CHBr—(CH₂)₁₃—CH₃ or —C(O)—(CH₂)₁₄—CH₃; —C(O)—CH₂—CH(OH)—COOH, —C(O)—CH₂—O—C(O)—CH₂CH(NH₂)—CONH₂, —C(O)—CH₂—O—CH₂CH₂OCH₃ or —C(O)—O—C(O)—CH₂CH₃.

An aliphatic group is a straight chained or branched hydrocarbon. Typically, a straight chained or branched alkyl group has from 1 to about 20 carbon atoms, preferably from 1 to about 10. Examples of alkyl groups include, but are not limited to, methyl, ethyl, n-propyl, iso-propyl, n-butyl, sec-butyl, tert-butyl, pentyl, hexyl, pentyl or octyl. A “lower alkyl” group is a C1-C8 straight chained or branched alkyl group.

The term “aromatic group” may be used interchangeably with “aryl,” “aryl ring,” “aromatic ring,” “aryl group” and “aromatic group.” Aromatic groups include carbocyclic aromatic groups such as phenyl, naphthyl, and anthracyl, and heteroaryl groups such as imidazolyl, thienyl, furanyl, pyridyl, pyrimidy, pyranyl, pyrazolyl, pyrroyl, pyrazinyl, thiazole, oxazolyl, and tetrazole. The term “heteroaryl group” may be used interchangeably with “heteroaryl,” “heteroaryl ring,” “heteroaromatic ring” and “heteroaromatic group.” Heteroaryl groups are aromatic groups that comprise one or more heteroatom, such as sulfur, oxygen and nitrogen, in the ring structure. Preferably, heteroaryl groups comprise from one to four heteroatoms.

Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

Non-aromatic heterocyclic rings are non-aromatic rings which include one or more heteroatoms such as nitrogen, oxygen or sulfur in the ring. The ring can be five, six, seven or eight-membered. Preferably, heterocyclic groups comprise from one to about four heteroatoms. Examples include tetrahydrofuranyl, tetrahyrothiophenyl, morpholino, thiomorpholino, pyrrolidinyl, piperazinyl, piperidinyl, and thiazolidinyl.

An acyl group is a group of the following formula “—C(O)R₁,” wherein R₁ is an alkyl. A lower acyl group is a group having the formula “—C(O)R₁,” wherein R₁ is a lower alkyl.

An alkyl group is a group of the following formula “—OR₁,” wherein R₁ is an alkyl. A lower alkoxy group is a group having the formula “—OR₁,” wherein R₁ is a lower alkyl.

Suitable substituents on an aliphatic group, non-aromatic heterocyclic group, or aryl group (carbocyclic and heteroaryl) are those which do not substantially interfere with the anti-cancer activity of the disclosed compounds. A substituent substantially interferes with anti-cancer activity when the anti-cancer activity is reduced by more than about 50% in a compound with the substituent compared with a compound without the substituent. Examples of suitable substituents include —R^{a}, —OH, —Br, —Cl, —I, —F, —OR^{a}, O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{c}COR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b})—NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}, —CR^{c}═CHR^{a}, —CR^{c}═CR^{a}R^{b}, —CCR^{a}, —SH, —SR^{a}, —S(O)R^{a},S(O)₂R^{a}

R^{a}-R^{d} are each independently an alkyl group, aromatic group, non-aromatic heterocyclic group or —N(R^{a}R^{b}), taken together, form a substituted or unsubstituted non-aromatic heterocyclic group. The alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}.

R^{#} is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂.

R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine. Optionally, the group —N(R⁺)₂ is a non-aromatic heterocyclic group, provided that non-aromatic heterocyclic groups represented by R+ and —N(R⁺)₂ that comprise a secondary ring amine are optionally acylated or alkylated.

Preferred substituents for a phenyl group, including phenyl groups represented by R₁-R₄, include C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN.

A paclitaxel analog can also be bonded to or be pendent from a pharmaceutically acceptable polymer, such as a polyacrylamide. One example of a polymer of this type is shown in FIG. 24. The term “paclitaxel analog”, as it is used herein, includes such polymers.

In some embodiments, paclitaxel anologs have a taxane skeleton represented by Structural Formula IX, wherein Z is O, S, or NR. Paclitaxel anologs that have the taxane skeleton shown in Structural Formula IX can have various substituents attached to the taxane skeleton and can have a double bond in zero, one or both of the cyclohexane rings as shown, for example in FIGS. 3-23.

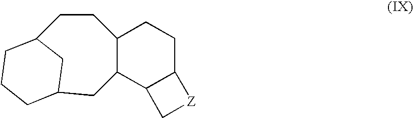

Various paclitaxel analogs and paclitaxel formulations that can be used in the method of the invention are described in Hennenfent et al. (2006) Annals of Oncology 17:735-749; Gradishar (2006) Expert Opin. Pharmacother. 7(8):1041-53; Attard et al. (2006) Pathol Biol 54(2):72-84; Straubinger et al. (2005) Methods Enzymol. 391:97-117; Ten Tije et al. (2003) Clin Pharmacokinet. 42(7):665-85; and Nuijen et al. (2001) Invest New Drugs. 19(2): 143-53, the entire teachings of which are incorporated herein by reference.

“Treating a subject with cancer” includes achieving, partially or substantially, one or more of the following results: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer. “Treating a subject with cancer” also includes partially or totally inhibiting, delaying or preventing the progression of cancer including cancer metastasis; partially or totally inhibiting, delaying or preventing the recurrence of cancer including cancer metastasis (in a subject who has been treated for cancer); or partially or totally preventing the onset or development of cancer (chemoprevention). Partially or totally inhibiting, delaying or preventing the recurrence of means inhibiting, delaying or preventing the recurrence of the cancer, after the original tumor has been removed, for example, by surgery. A subject who has been “treated for cancer”, is a subject in which, for example, the primary tumor has been, for example, removed surgically or has gone into remission following treatment by, for example, chemotherapy.

An “effective amount” is the quantity of an agent or agents in which a beneficial clinical outcome is achieved when the agent or agents are administered to a subject. For example, when, an anti-cancer therapy, such as a chemotherapeutic agent, is administered with an agent that increase the oxidative stress of a cell, a “beneficial clinical outcome” includes a reduction in tumor mass, a reduction in metastasis, a reduction in the severity of the symptoms associated with the cancer and/or an increase in the longevity of the subject compared with the absence of the treatment. The precise amount of the chemotherapeutic agent or the compound that induces oxidative stress in a cell administered to a subject will depend on the type and severity of the disease or condition and on the characteristics of the subject, such as general health, age, sex, body weight and tolerance to drugs. It may also depend on the degree, severity and type of cancer. The skilled artisan will be able to determine appropriate dosages depending on these and other factors.

A compound increases the efficacy of another anti-cancer therapy when they are administered in combination if it increase the beneficial clinical outcome achieved by the anti-cancer agent alone or achieves the same beneficial clinical outcome of the anti-cancer agent alone at a lower dose of the anti-cancer agent.

As used herein, the terms “subject”, “patient” and “mammal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

### II. Description of Embodiments of the Invention

In one aspect, the invention relates to a compound that increases oxidative stress in a cell, wherein the compound increases p38 activity. The signaling protein Daxx binds to the Fas receptor, a cell-surface protein receptor expressed on essentially all cells of the body that when bound to its ligand (FasL) signals a caspase cascade, ultimately resulting in apoptosis. When bound to the Fas receptor, Daxx interact with apoptosis signal-regulating kinase (ASK1). ASK1 activates MAP kinase kinase (MAPKK), SEK1 (or MKK4), and MKK3/MAPKK6 (or MKK6), which in turn activate JNK/SAPK and p38 leading to apoptosis. Apoptosis by this pathway can be mediated directly by the redox state of a cell. The redox state of a cell is the consequence of a balance between levels of ROS and reducing equivalents (e.g., small proteins with redox-active thiol groups such as glutathione and thioredoxin). When a cell in not under oxidative stress, the cellular stores of reducing equivalents is large enough to neutralize ROS that are generated by metabolic processes of the cell. However, when a cell is contacted with a compound that induces oxidative stress, the number of ROS generated by the cell out ways the cellular store of reducing equivalents and oxidative stress occurs. ASK1 associates with thioredoxin and inactives it when a cell in not under oxidative stress. However, when thioredoxin becomes oxidized it releases ASK1 which can then signal apoptosis through the p38 or JNK/SAPK signaling pathway.

In one embodiment, compounds of the invention increase the activity of ASK1. In another embodiment, compounds of the invention disrupt the association of ASK1 and thioredoxin.

In some embodiments, compound that increases oxidative stress and p38 activity in a cell also induces the production of Hsp70. Preferably, the compound induces the production of Hsp70 to a greater extent cancer cells compared to normal cells. For example, the compound induces the production of Hsp70 2-fold, 5-fold, 10-fold, 100-fold, 200-fold or more in cancer cells compared to non-cancerous cells. In some embodiments, the compound increases the amount of membrane-bound Hsp70 in cancer cells after the administration of the compound compared to the membrane-bound Hsp70 in the cancer cells before administration. In some embodiments, the compound increase the amount of Hsp70 secreted by the cancer cells after the administration of the compound compared to the Hsp70 secreted by the cancer cells before administration. In some embodiments, the compound does not significantly inhibit the activity of Hsp90. In some embodiments, the compound increase production of granzyme B in natural killer cells that contact the cancerous cells. In some embodiments, the cancer is a cancer responsive to immunotherapy, such as melanoma, renal cell carcinoma, pancreatic cancer or bladder cancer. In some embodiments, compounds of the invention do not include compounds disclosed in the patents and patent applications listed in Table 1.

In another aspect, the invention relates to a method of identifying a compound that has synergistic anticancer activity with a taxane, comprising the steps of:
a) determining Hsp70 expression in the cell before contacting the cell with a test compound;
b) contacting the cell with the test compound; and
c) determining Hsp70 expression in the cell after contact with the test compound, wherein increased levels of Hsp70 expression in the cell after contact with the test compound indicates that the compound has synergistic anticancer activity with a taxane.
   In one embodiment, the method of identifying a compound that has synergistic anticancer activity with a taxane, further comprising the steps of:
d) determining the activity of p38 before the cell is contacted with the test compound; and
e) determining the activity of p38 after the cell has been contacted with the test compound, wherein increased activity of p38 in the cell after contact with the test compound indicates that the compound has synergistic anticancer activity with a taxane.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with cancer. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in treating cancer. In addition, a synergistic effect can result in improved efficacy of agents in the treatment of cancer. Finally, a synergistic effect of a combination of therapies (e.g., a combination of prophylactic or therapeutic agents) may avoid or reduce adverse or unwanted side effects associated with the use of either therapy alone.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

In another aspect, the invention relates to a method of treating cancer in a subject, comprising the steps of:
a) administering to the subject an anti-cancer therapy; and
b) administering to the subject a compound that increases oxidative stress in the cancer cells and increases p38 activity, provided that the compound is not a compound having the following structural formula:

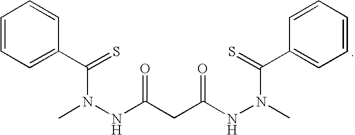

In one embodiment, the anti-cancer therapy is a chemotherapeutic agent. Preferably, the chemotherapeutic agent is paclitaxel or a paclitaxel analog. In another preferred embodiment, the chemotherapeutic agent is an immunotherapy.

In some embodiments, the cancer therapy and the compound that increases oxidative stress and activates p38 are administered simultaneously, preferably in the same pharmaceutical formulation. In another embodiment, the anti-cancer therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the compound that increases oxidative stress to a subject with cancer.

In some embodiments, the invention relates to a pharmaceutical composition, comprising a pharmaceutically acceptable carrier and a compound that increases oxidative stress and p38 activity, or a pharmaceutically acceptable salt thereof. In some embodiments, the compound that increases oxidative stress and p38 activity induces the production of Hsp70 to a greater extent in cancer cells compared to normal cells. For example, the compound induces the production of Hsp70 2-fold, 5-fold, 10-fold, 100-fold, 200-fold or more in cancer cells compared to non-cancerous cells. In some embodiments, the compound increases the amount of membrane-bound Hsp70 in cancer cells after the administration of the compound compared to the membrane-bound Hsp70 in the cancer cells before administration. In some embodiments, the compound increases the amount of Hsp70 secreted by the cancer cells after the administration of the compound compared to the Hsp70 secreted by the cancer cells before administration. In some embodiments, the pharmaceutical composition further comprises a chemotherapeutic agent. In some embodiments, the chemotherapeutic agent is paclitaxel or a paclitaxel analog. In some embodiments, the compound that increases oxidative stress and p38 activity does not significantly inhibit the activity of Hsp90. In some embodiments, the compound that increases oxidative stress and p38 activity increases production of granzyme B in natural killer cells that contact the cancerous cells. In some embodiments, the compound that increases oxidative stress and p38 activity is not a compound disclosed in the patents and patent applications listed in Table 1.

Suitable pharmaceutically acceptable carriers may contain inert ingredients which do not inhibit the biological activity of the agent. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, Mack Publishing Company, Easton, Pa. Formulation of the compound to be administered will vary according to the route of administration selected (e.g., solution, emulsion, capsule). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrins) are known in the art (Baker, et al., “Controlled Release of Biological Active Agents”, John Wiley and Sons, 1986).

The agents are administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds of the invention can also be administered orally (e.g., dietary), topically, by inhalation (e.g., intrabronchial, intranasal, oral inhalation or intranasal drops), or rectally, depending on the type of cancer to be treated. Oral and parenteral administrations are preferred modes of administration.

Cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

Other examples of leukemias include acute and/or chronic leukemias, e.g., lymphocytic leukemia (e.g., as exemplified by the p388 (murine) cell line), large granular lymphocytic leukemia, and lymphoblastic leukemia; T-cell leukemias, e.g., T-cell leukemia (e.g., as exemplified by the CEM, Jurkat, and HSB-2 (acute), YAC-1 (murine) cell lines), T-lymphocytic leukemia, and T-lymphoblastic leukemia; B cell leukemia (e.g., as exemplified by the SB (acute) cell line), and B-lymphocytic leukemia; mixed cell leukemias, e.g., B and T cell leukemia and B and T lymphocytic leukemia; myeloid leukemias, e.g., granulocytic leukemia, myelocytic leukemia (e.g., as exemplified by the HL-60 (promyelocyte) cell line), and myelogenous leukemia (e.g., as exemplified by the K562 (chronic)cell line); neutrophilic leukemia; eosinophilic leukemia; monocytic leukemia (e.g., as exemplified by the THP-1 (acute) cell line); myelomonocytic leukemia; Naegeli-type myeloid leukemia; and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B.C. Decker Inc. (2000). The entire teachings of the preceding references are incorporated herein by reference.

In one embodiment, the disclosed method is believed to be particularly effective in treating a subject with non-solid tumors such as multiple myeloma. In another embodiment, the disclosed method is believed to be particularly effective against T-leukemia (e.g., as exemplified by Jurkat and CEM cell lines); B-leukemia (e.g., as exemplified by the SB cell line); promyelocytes (e.g., as exemplified by the HL-60 cell line); uterine sarcoma (e.g., as exemplified by the MES-SA cell line); monocytic leukemia (e.g., as exemplified by the THP-1 (acute) cell line); and lymphoma (e.g., as exemplified by the U937 cell line).

The disclosed method is particularly effective at treating subjects whose cancer has become “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. Drug resistant tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

In another embodiment, the disclosed method is believed to be particularly effective in treating a subject with an immunosensitive cancer. Immunosensitive cancers are cancers that respond to treatment with immunotherapy. Immunotherapy is described below in more detail. Cancers that respond to immunotherapy include renal cell carcinoma, pancreatic cancer, bladder cancer, melanoma (including superficial spreading melanoma, nodular melanoma, acral lentiginous melanoma, lentigo maligna melanoma which is also called Hutchinson's Freckle), multiple myeloma, myeloma, lymphoma, non-small-cell lung cancer, squamous cell carcinoma, basal cell carcinoma, fibrosarcoma and malignant brain tumors.

In another embodiment, the disclosed method is believed to be particularly effective in treating a subject with melanoma.

In another embodiment, the disclosed method is believed to be particularly effective in treating a subject with renal cell carcinoma.

In another embodiment, the disclosed method is believed to be particularly effective in treating a subject with pancreatic cancer.

In another embodiment, the disclosed method is believed to be particularly effective in treating a subject with bladder cancer.

Immunotherapy (also called biological response modifier therapy, biologic therapy, biotherapy, immune therapy, or biological therapy) is treatment that uses parts of the immune system to fight disease. Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body. In a preferred embodiment, a compound that increases oxidative stress and p38 activity may be administered in combination with an immunotherapy to treat cancer, preferably to treat an immunosensitive cancer.

Examples of active immunotherapies include: cancer vaccines, tumor cell vaccines (autologous or allogeneic), dendritic cell vaccines, antigen vaccines, anti-idiotype vaccines, DNA vaccines, Lymphokine-Activated Killer (LAK) Cell Therapy, or Tumor-Infiltrating Lymphocyte (TIL) Vaccine with Interleukin-2 (IL-2). Active immunotherapies are currently being used to treat or being tested to treat various types of cancers, including melanoma, kidney (renal) cancer, ovarian cancer, breast cancer, colorectal cancer, lung cancer, leukemia, prostate cancer, non-Hodgkin's lymphoma, pancreatic cancer, lymphoma, multiple myeloma, head and neck cancer, liver cancer, malignant brain tumors, and advanced melanoma.

Examples of passive immunotherapies include: monoclonal antibodies and targeted therapies containing toxins. Monoclonal antibodies include naked antibodies and conjugated antibodies (also called tagged, labeled, or loaded antibodies). Naked monoclonal antibodies do not have a drug or radioactive material attached whereas conjugated monoclonal antibodies are joined to a chemotherapy drug (chemolabeled), a radioactive particle (radiolabeled), or a toxin (immunotoxin). A number of naked monoclonal antibody drugs have been approved for treating cancer, including:

Rituximab (Rituxan), an antibody against the CD20 antigen used to treat B cell non-Hodgkin lymphoma; Trastuzumab (Herceptin), an antibody against the HER2 protein used to treat advanced breast cancer; Alemtuzumab (Campath), an antibody against the CD52 antigen used to treat B cell chronic lymphocytic leukemia (B-CLL); Cetuximab (Erbitux), an antibody against the EGFR protein used in combination with irinotecan to treat advanced colorectal cancer and to treat head and neck cancers; and Bevacizumab (Avastin) which is an antiangiogenesis therapy that works against the VEGF protein and is used in combination with chemotherapy to treat metastatic colorectal cancer. A number of conjugated monoclonal antibodies have been approved for treating cancer, including: Radiolabeled antibody Ibritumomab tiuxetan (Zevalin) which delivers radioactivity directly to cancerous B lymphocytes and is used to treat B cell non-Hodgkin lymphoma; radiolabeled antibody Tositumomab (Bexxar) which is used to treat certain types of non-Hodgkin lymphoma; and immunotoxin Gemtuzumab ozogamicin (Mylotarg) which contains calicheamicin and is used to treat acute myelogenous leukemia (AML). BL22 is a conjugated monoclonal antibody currently in testing for treating hairy cell leukemia and there are several immunotoxin clinical trials in progress for treating leukemias, lymphomas, and brain tumors. There are also approved radiolabeled antibodies used to detect cancer, including OncoScint for detecting colorectal and ovarian cancers and ProstaScint for detecting prostate cancers. Targeted therapies containing toxins are toxins linked to growth factors and do not contain antibodies. An example of an approved targeted therapy containing toxins is denileukin diftitox (Ontak) which is used to treat a type of skin lymphoma (cutaneous T cell lymphoma).

In one embodiment, the invention relates to a method of preventing the reoccurrence of cancer in a subject, comprising administering to the subject an anti-cancer therapy (adjuvant therapy) and administering to the subject a compound that increases oxidative stress and p38 activity. In one embodiment, the adjuvant therapy is an immunotherapy. Examples of adjuvant immunotherapies include: cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guérin (BCG); Keyhole limpet hemocyanin (KLH); Incomplete Freund's adjuvant (IFA); QS-21; DETOX; Levamisole; and Dinitrophenyl (DNP). Clinical studies have shown that combining IL-2 with other cytokines, such as IFN-alpha, can lead to a synergistic response.

Several types of immunotherapies are being used to treat melanoma patients. IFN-alpha and IL-2 are approved for treatment of people with metastatic melanoma. BCG is being tested in combination with melanoma vaccines and other immunotherapies. Tumor-infiltrating lymphocytes have been shown to shrink melanoma tumors in a phase 1 clinical trial. Human monoclonal antibodies to ganglioside antigens have been shown to regress cutaneous recurrent melanoma tumors. Some autologous and allogeneic tumor cell vaccines, antigen vaccines (including polyvalent antigen vaccines), and dendritic cell vaccines have also been shown to shrink tumors. Clinical trials continue for these and other melanoma immunotherapies. Melanoma patients with a high IgM response often survive better than those who elicit no or low IgM antibodies (Morton et al., 1992). Combined IL-12/TNF-alpha immunotherapy has been shown to significantly retard tumor growth in three tumor models in mice (B16F10 melanoma, Lewis lung (LL/2) carcinoma and L1 sarcoma) as compared with controls and mice treated with either cytokine alone. IFN-alpha is approved for the treatment of malignant melanoma, chronic myelogenous leukemia (CML), hairy cell leukemia, and Kaposi's sarcoma. In one embodiment, the invention relates to a method of treating melanoma in a subject, comprising administering to the subject one or more of the above immunotherapies for treating melanoma and administering to the subject a compound that increases oxidative stress and p38 activity.

Several types of immunotherapies are being used to treat patients that have renal cell carcinoma. IFN-alpha and IL-2 are approved for treatment of people with metastatic renal (kidney) cancer. A combination therapy using IL-2, interferon, and chemotherapy is being tested for treatment of renal cell carcinoma. Treatment with a tumor cell vaccine plus the adjuvant BCG has been shown to shrink tumors in some advanced renal cell carcinoma patients. DNA vaccines and tumor-infiltrating lymphocytes are also being tested as treatments for renal cell carcinoma. Chimeric bispecific G250/anti-CD3 monoclonal antibodies have been shown to mediate cell lysis of renal cell carcinoma cell lines by cloned human CD8+ T cells or by IL-2 stimulated peripheral blood lymphocytes. In one embodiment, the invention relates to a method of treating renal cell carcinoma in a subject, comprising administering to the subject one or more of the above immunotherapies for treating renal cell carcinoma and administering to the subject a compound that increases oxidative stress and p38 activity.

### EXEMPLIFICATION

### Example 1

### Compound 1 Preferentially Induces Hsp70 in MDA-MB-435 Cells Compared to Non-Cancerous Epithelial Cells

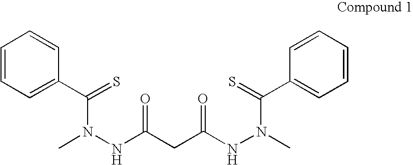

MDA-MB-435, human mammary epithelial (HMEC) which has a pattern of gene expression that more closely resembled that of melanoma cell lines than of other breast tumor lines (see Ross et al., “Systematic variation in gene expression patterns in human cancer cell lines,” Nat. Genet. (2000). 24(3):227-3.), and human renal epithelial (HRE) cells were seeded in 100 mm tissue culture plates at 1.5×10⁶ cells/plate and incubated at 37° C. with 5% CO₂ for several hours. The cells were then treated with 0.5 μM of Compound 1 in DMSO or with DMSO alone at 37° C. for 15 hours. After treatment the cells were lysized and used for Western blot analysis.

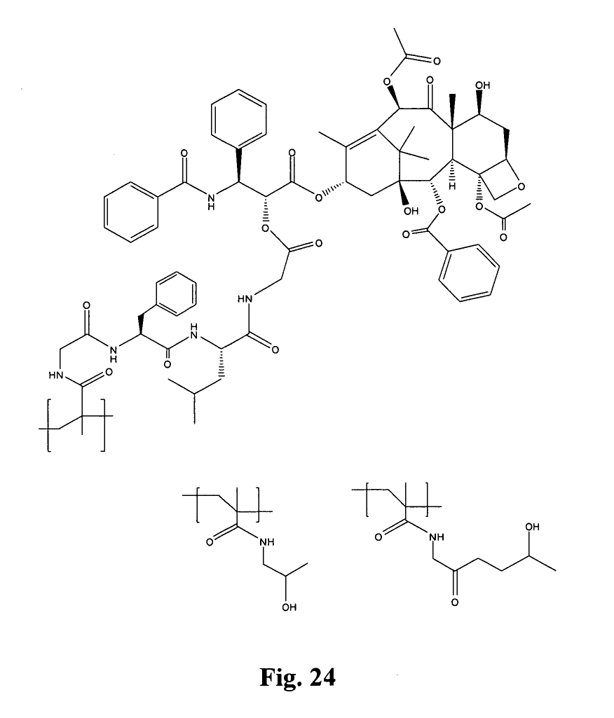

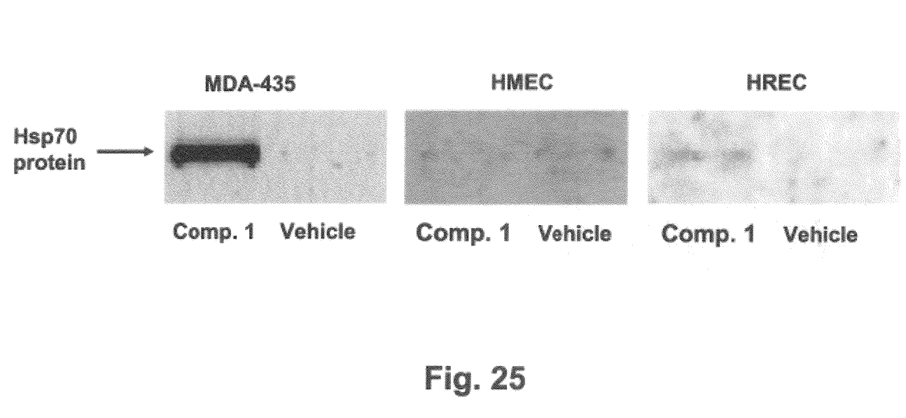

Samples were run on a SDS-PAGE gel and proteins were transferred onto a nitrocellulose membrane. Nitrocellulose was blocked with 5% skim milk in TBS with 0.5% Tween at room temperature for 1 hour and then probed with anti-Hsp70 (mouse IgG, sc-24, Santa Cruz) antibody. The nitrocellulose was then probed with secondary antibodies, HRP-conjugated goat anti-rabbit IgG and HRP-conjugated horse anti-mouse IgG (#7074, #7076, Cell Signaling). LumiGLO reagent, 20× Peroxide (#7003, Cell Signaling) was used to visualize gel (see FIG. 25). FIG. 25 shows that Compound 1 preferentially induces Hsp70 in MDA-MB-435 tumor cells compared to non-tumor HMEC, and HRE cells.

### Example 2

### Compound 1 Preferentially Sensitizes MDA-MB-435 Cells to Lysis by Mouse Splenocytes Compared to Non-Cancerous Epithelial Cells

MDA-MB-435 cells were purchased from American Type culture collection. Human mammary epithelial (HMEC), human renal epithelial (HRE) cells and medium for maintaining the cells were obtained from Cambrex Bioproducts. Mouse splenocytes were isolated form CD1 nude mice (Jackson Lab).

MDA-MB-435, HMEC and HRE cells were seeded (3×10³ cells/well) in 96-well plates and incubated at 37° C. with 5% CO₂ for several hours. The cells were then treated with 0.1 or 0.5 μM of Compound 1 in DMSO or with DMSO alone and incubated at 37° C. for about 15 hours. Mouse splenocytes were isolated from Nude mice and mixed with MDA-MB-435, HMEC and HRE cells at ratio of 10:1. The mixed cells were incubated at 37° C. for 24 hours in the tissue culture medium and MTS assay was performed to measure viability of the cells with an ELISA reader (DYNEX Technologies).

Cytotoxicity (C) was calculated based on an equation below:

C=[1−(OD_{MIX}−OD_{EFF})/(OD_{TAR}−OD_{MED})]×100%

Wherein:

OD_{MIX} is optical density of the wells containing mixture of effector (mouse splenocyte) and target cells (MDA-MB-435, HMEC and HRE cells);

OD_{EFF} is optical density of the wells with effector cells;

OD_{TAR} is optical density of the wells with target cells; and

OD_{MED} is optical density of the wells with medium without cells.

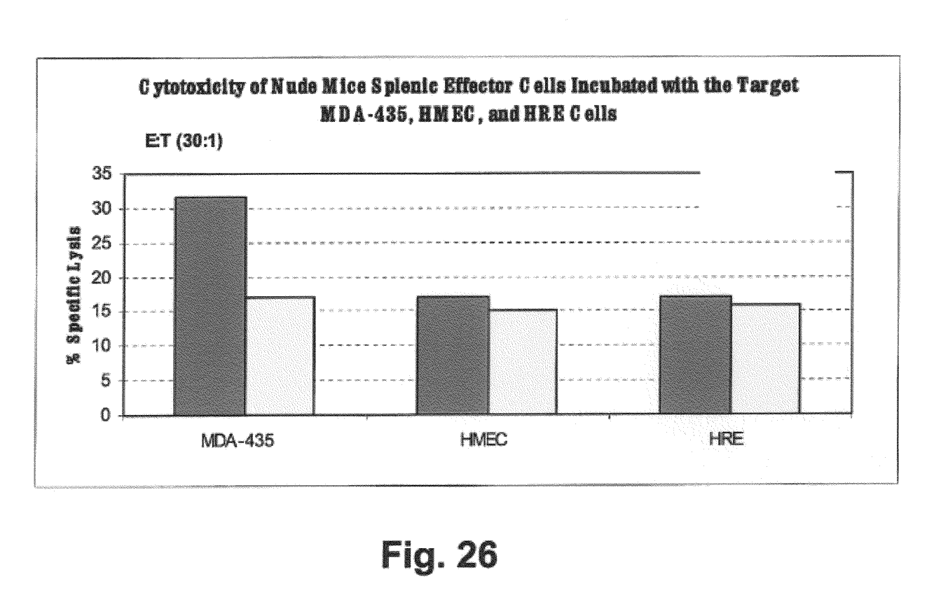

FIG. 26 shows the percent lysis of target cells that had been treated with Compound 1 (blue bars) compared to cells which were treated with vehicle alone (yellow bars). Treatment with Compound 1 increased lysis of MDA-MB-435 tumor cell line by about 15% compared to MDA-MB-435 cells which were treated with DMSO alone. Treatment with Compound 1 only slightly increased the lysis of non-cancerous HMEC and HRE cells compared to cells treated with DMSO alone.

### Example 3

### Compound 1 Induces an Oxidative Stress Signature Profile in MDA-MB-435 Cells

1×10⁶ Hs294T (or MDA-MB-435) cells (ATCC Cat. # HTB-140, HTB-129) were plated then treated 3 hours later with DMSO as a control or Compound 1 (100 nM). RNA was isolated from the cells 6 hours after treatment using the RNeasy kit according to the manufacturers instructions (Qiagen Cat. # 71404) and RNA quality was confirmed by spectrophotometric examination. cDNA was synthesized using the One-Cycle cDNA Synthesis Kit (Affymetrix Cat. # 900431) according to the manufacturers instructions. cDNA was labeled using the GeneChip IVT Labeling Kit (Affymetrix Cat. #900449) and analyzed in duplicate on the Affymetrix Human Genome U133Plus 2.0 gene chip array according to standard Affymetrix protocols. Data sheets from the Affymetrix Gene Chip Scanner 3000 were imported into Microsoft Excel and filtered by the following criteria: greater than or equal to 1.5 fold increase, greater than or equal to 0.4SE between replicates; and 50 (low stringency) or 500 (high stringency) for the lowest signal.

Compound 1 increase the transcription of Hsp70 by over 300 fold. Compound 1 also increases the transcription of the following additional heat shock proteins: Hsp105, Hsp90, Hsp60, Hsp47, Hsp40, Hsp27, Hsp10, crystallin alpha B, AHA1 and BAG3; the following metallothioneins: MT1E, MT1F, MT1G, MT1H, MT1M, MT1X and MT2A; and the following antioxidants: transaldolase 1, superoxide dismutase 2, thioredoxin reductase 1, peroxiredoxin 1, glutamate cysteine ligase, and glutathione reductase 1. These results indicate that Compound 1 induces an oxidative stress signature profile in MDA-MB-435 cells.

To determine whether oxidative stress mediates the entire gene profile induced by Compound 1, a transcriptional profiling experiment was performed on cells treated with Compound 1 (100 nM) plus the antioxidant N-acetyl-cysteine (NAC) (Sigma) (5 mM). NAC is the rate-limiting amino acid for the production of the antioxidant glutathione. Addition of NAC to cells increases the levels of endogenous glutathione which in turn blocks oxidative stress and ROS generation. Hs294 T cells were plated for 3 hours and then NAC was added to a final concentration of 5 mM for 30 minutes prior to treatment with Compound 1 (100 nM). RNA was isolated from the cells 6 hours after treatment and Affymetrix analysis was performed as above.

As observed above, Compound 1 robustly induced heat shock and metallothionein genes by 6 hours in Hs294T melanoma cells. Addition of NAC to Compound 1 treated cells inhibited the genes most robustly induced by Compound 1. This result suggests that oxidative stress is responsible for the induction of most genes by Compound 1.

### Example 4

### Compound 1 Induces Cell Surface Expression of Hsp70

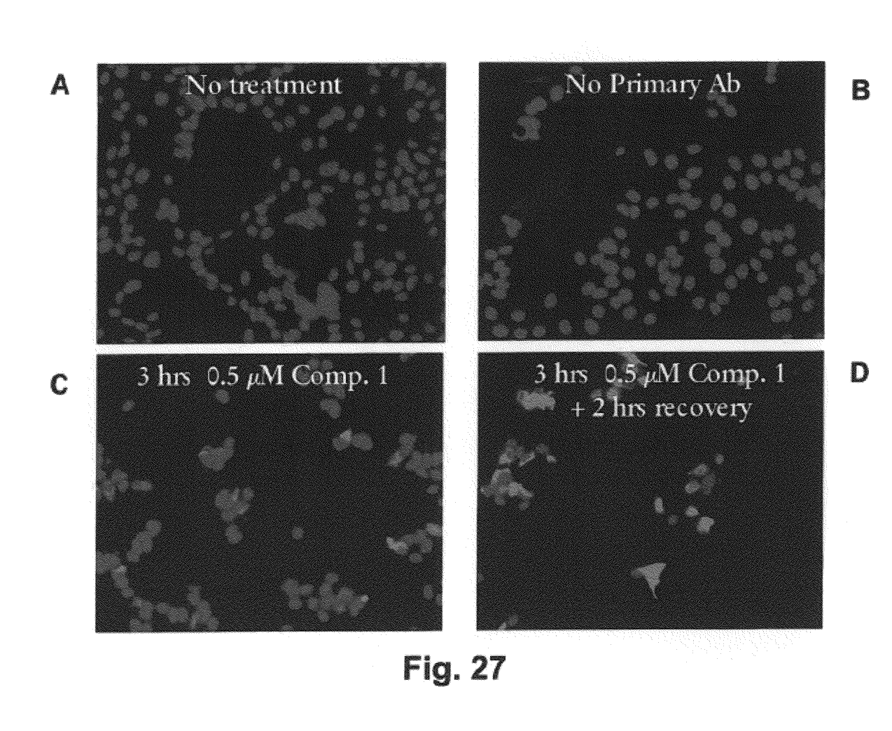

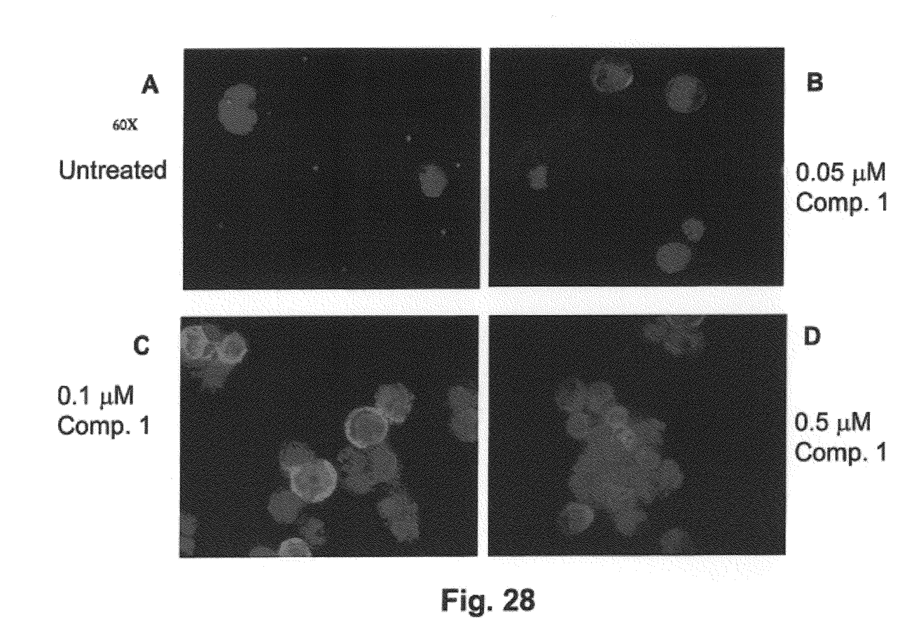

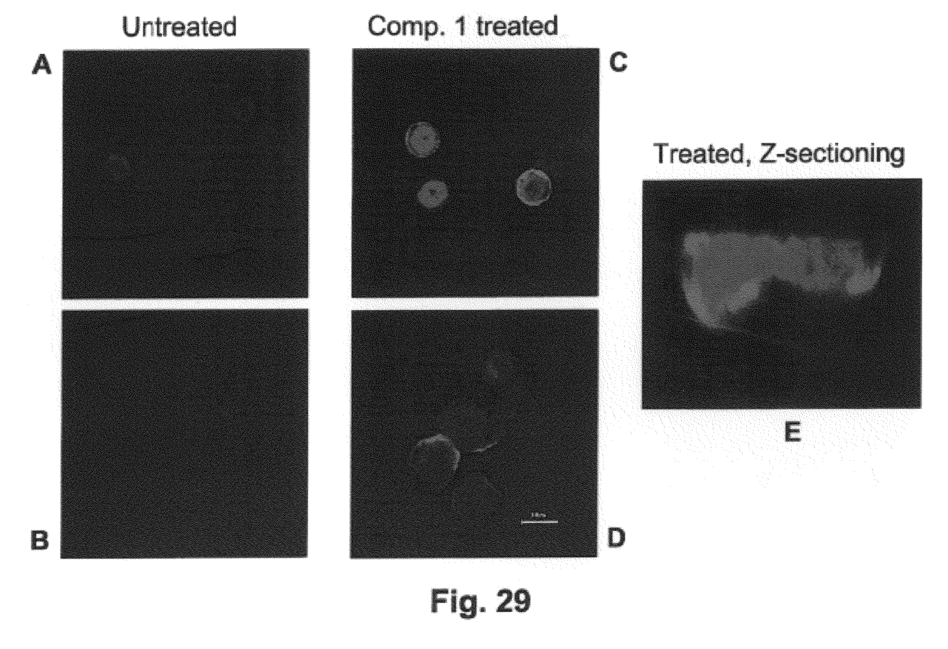

MCF-7 (derived from human breast adenocarcinoma), CEM (derived from T cell leukemia) or HT-29 (derived from human colon adenocarcinoma) cells were cultured in 10% FBS containing DMEM medium for 24 hrs and treated with DMSO or Compound 1 at various concentration for various times. After treatment, cells were rinsed one time in PBS then fixed in 2% Paraformaldehyde at room temperature for 20 minutes. Slides with the fixed cells were rinsed with PBS two times. Primary antibody mouse monoclonal anti-Hsp70 (Stressgen SPA-8 10) was diluted 1:100 in PBS and the slides were incubated 1 hour at 37° C. The slides were rinsed two times with PBS. Secondary antibody anti-mouse AlexaFluor 488 (Molecular Probes A-11001) was diluted 1:500 in PBS and the slides were incubated 30 min at 37° C. The slides were rinsed two times in PBS. DAPI (Molecular Probes D-3571) were diluted 1:10,000 in PBS and the slides were incubated at room temperature in dark for 5 min. The slides were rinsed two times in PBS, then once in deionized water and left to dry in dark until almost completely dry. Coverslips were mounted with Prolong Anti-fade Mounting Media (Molecular Probes P-748 1). The images were captured with a monochrome CCD camera SenSys (see FIGS. 27A-D and 28A-D) or by confocal microscopy (see FIG. 29A-E). In FIGS. 27A-D and 28A-D, the bright pink color on the surface of the MCF-7 cells (FIG. 27A-D) or CEM cells (FIG. 28A-D) indicates Hsp70 on the surface of the cells. In FIG. 29A-E the bright green color on the surface of the cell indicates Hsp70 on the surface of the HT29 cells. As can be seen from FIGS. 27A-29E, cells which were untreated show much less Hsp70 on their surface than cells which were treated with Compound 1.

### Example 5

### Compound 1 Induces the Shedding of Hsp70 in Cultured Tumor Cells

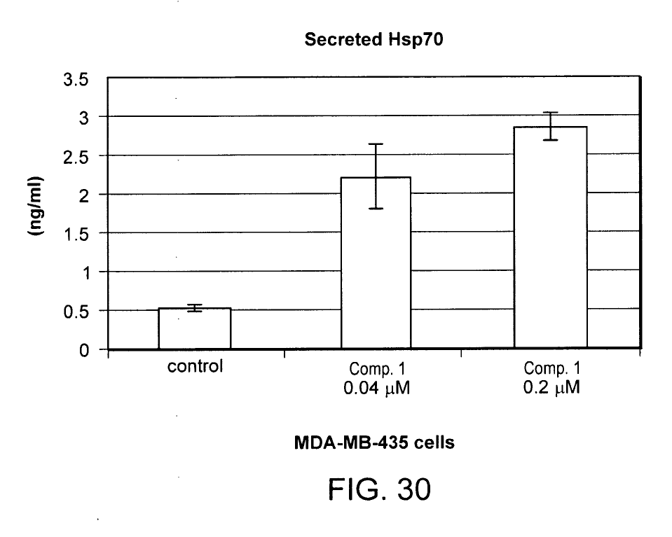

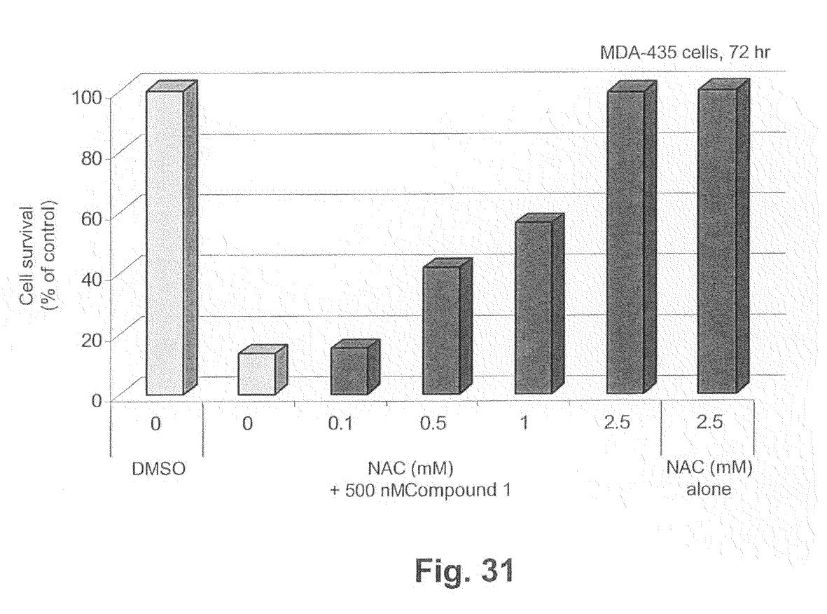

MDA-MB-435 tumor cells were seeded at 15,000 cells/150 μl/well of 48-well plate and cultured in DMEM supplemented with 10% fetal bovine serum. The compounds were dissolved in dimethyl sulfoxide (DMSO) then a solution was prepared containing 0.04 μM or a 0.2 μM in 25% DMSO and 75% culture medium. Cells were treated with either the 0.04 μM or the 0.2 μM solution of Compound 1 or with a solution of 25% DMSO/75% culture medium for 72 h. The medium was then separated from the cells and Hsp70 was measured by ELISA kit from Stressgen. Cell viability was checked by CCK-8 cell count assay (Dojindo) at 88 hr. FIG. 30 is a graph showing the amount of Hsp70 secreted by cells treated with Compound 1 or untreated. As can be seen from FIG. 30, Compound 1 increased the secretion of Hsp70 by the cells in a dose dependent manner.

### Example 6

### Apoptosis Induced by Compound 1 is Inhibited by the Antioxidant NAC

To examine whether the antioxidant N-acetyl-cysteine (NAC) inhibits Compound 1 induced apoptosis in a dose dependent manner, MDA-MB-435 cells were seeded in a 96-well plate at 5000 cells/well and incubated at 37° C. for 4 hours. The cells were pretreated without or with NAC at different concentrations for 30 minutes and then treated without or with 500 nM Compound 1. The cells were further cultured at 37° C. for 72 hours and cell viability was measured with Cell Counting Kit-8 (Dojindo Laboratories, Cat# CK04).

Exposure of MDA-MB-435 melanoma cells to 500 nM Compound 1 reduced cell viability at 72 hours post-drug treatment by approximately 90% (determined in an MTT assay). Treatment of Compound 1 exposed cells with increasing concentrations of NAC blocked Compound 1-induced cell death in a dose-dependent manner. Indeed, 2.5 mM NAC completely blocked the ability of Compound 1 to induce cell death (see FIG. 31)

To confirm that the ability of NAC to block Compound 1-induced cell death was not cell-line specific, we examined the role of oxidative stress in two additional cell lines by FAC analysis. RAMOS and HSB2 cells (ATCC) were seeded in 6-well plates at 1×10⁶ cells/well/4 mL and incubated at 37° C. for 4 hours. The cells were pretreated without or with NAC at 5 mM for one hour and then treated without or with Compound 1 for 24 hours. After incubation the cells were stained with Annexin V and propidium iodide (Vybrant Apoptosis assay kit, Cat# V13241, Invitrogen, Molecular Probes) for measuring apoptotic cells by FACS analysis.

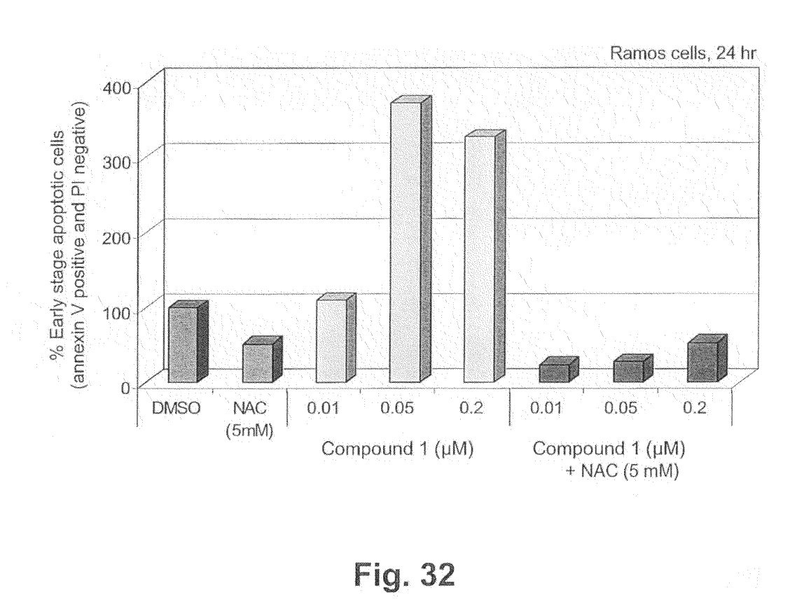

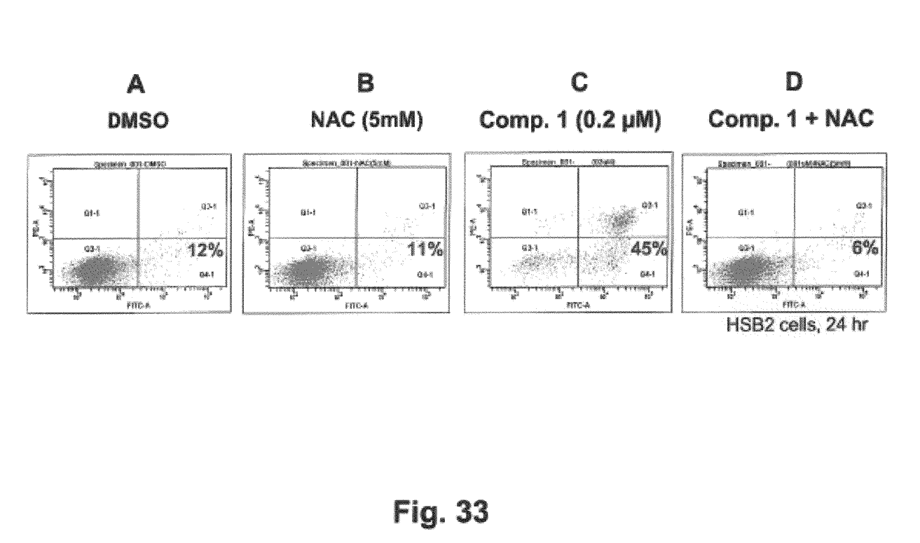

Exposure of the Ramos B cell lymphoma cell line to increasing concentrations of Compound 1 increased the number of early apoptotic cells (annexin V positive, PI negative) relative to DMSO treated cells (FIG. 32). Treatment of Compound 1 exposed cells to 5 mM NAC completed inhibited early apoptotic signaling. Similarly, exposure of the HSB2 T cell leukemia cell line to 200 nM Compound 1 increased the number of early apoptotic cells from 12% (DMSO) (FIG. 33A) to 45% (Compound 1) (FIG. 33C). Treatment with 5 mM NAC in combination with Compound 1 (FIG. 33D) completely blocked the increase in early apoptotic cells. These data demonstrate that drug-induced oxidative stress mediates apoptotic cell death in multiple cell lines.

### Example 7

### Hsp70 Induction by Compound 1 is Inhibited by NAC

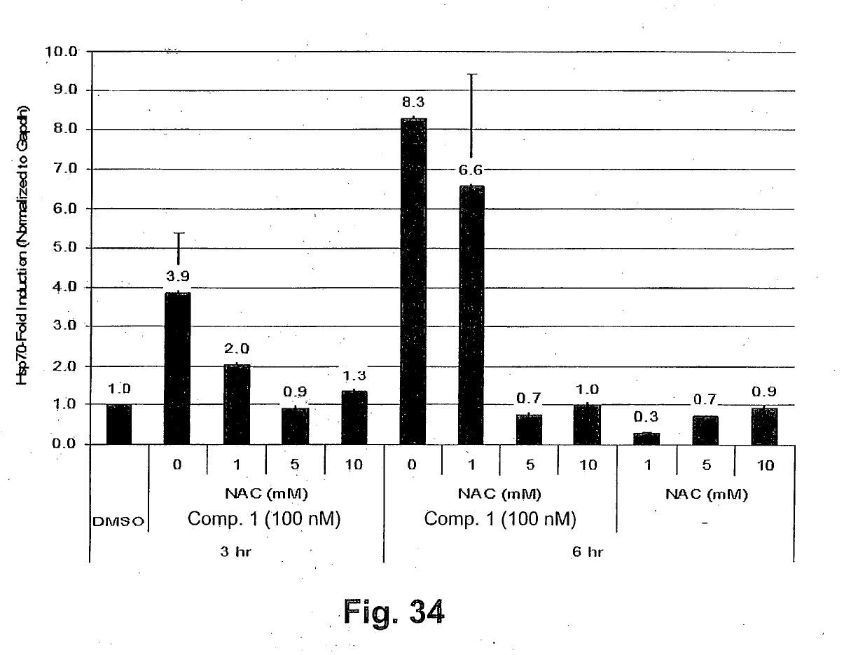

We examined the effects of NAC treatment on the ability of Compound 1 to induce Hsp70 RNA induction by quantitative PCR analysis (FIG. 34). Exposure of human melanoma Hs294T cells to 100 nM Compound 1 for 3 and 6 hours induced Hsp70 RNA expression 3.9 and 8.3 fold, respectively. Hs294 T cells were plated for 3 hours and then NAC was added to a final concentration of 5 mM for 30 minutes prior to treatment with Compound 1 (100 nM). RNA was isolated from the cells 6 hours after treatment. 500 ng of RNA from each sample was used for cDNA synthesis using iScript cDNA Synthesis Kit (Biorad, Cat# 170-8891). 1 uL of cDNA mixture was used with the iQ SYBR Green Supermix (Biorad, Cat# 170-8882) to perform quantitative PCR in an iCycler machine (Biorad) using standard protocols. QPCR for each sample/time point was performed in duplicate. The following primers for inducible human Hsp70 and gapdh were used:

Hsp70-F3 5′-AAG-GAC-ATC-AGC-CAG-AAC-AAG-CG-3′
Hsp70-R3 5′-AAG-AAG-TCC-TGC-AGC-AGC-TTC-TGC-3′
Gapdh-F2 5′-AAG-GTC-GGA-GTC-AAC-GGA-TTT-GGT-3′
Gapdh-R2 5′-CAT-GGT-TCA-CAC-CCA-TGA-CGA-ACA-3′

Ct values were obtained from the iCycler program and used in an equation (2^{-ΔΔC}T) for determining the amount of target relative to gapdh expression. This compares the relative expression of Hsp70 to the corresponding levels of gapdh in each sample. Duplicate wells were averaged and graphed with their corresponding standard deviation.

Treatment of Compound 1 exposed cells with increasing concentrations of NAC inhibited Hsp70 induction in a dose-dependant manner. At 5 mM NAC, a complete inhibition in Hsp70 induction was observed. Thus, oxidative stress and ROS generation are likely key signals that cause Hsp70 induction in response to Compound 1 exposure.

### Example 8

### Compound 1 Activates p38 Kinase

MDA-MB-435 tumor cells were either untreated or treated with 10 nM, 50 nM, 100 nM, 500 nM or 1000 nM of Compound 1 in DMSO for 4 hours. After treatment the cells were lysized and used for Western blot analysis.

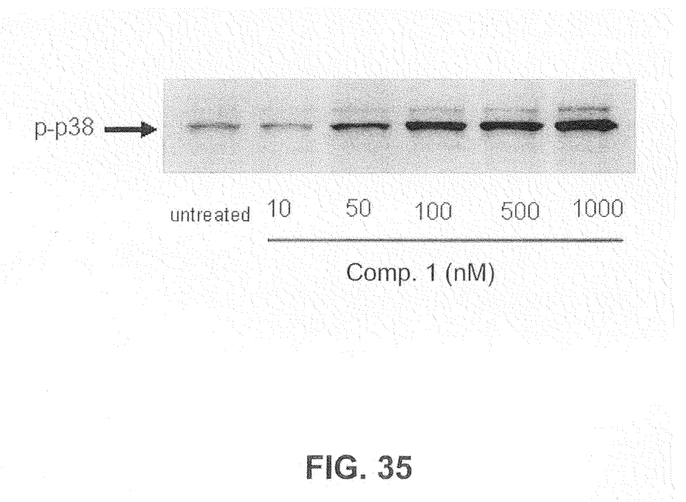

Samples were run on a SDS-PAGE gel and proteins were transferred onto a nitrocellulose membrane by a semi-dry transfer. Nitrocellulose was blocked with 5% skim milk in TBS with 0.5% Tween at room temperature for 1 hour and then probed with anti-phospho-p38 (Cell Signaling, #9216) antibody. The nitrocellulose was then probed with secondary antibodies, HRP-conjugated goat anti-rabbit IgG and HRP-conjugated horse anti-mouse IgG (#7074, #7076, Cell Signaling). LumiGLO reagent, 20× Peroxide (#7003, Cell Signaling) was used to visualize gel (see FIG. 35). As can be seen in FIG. 35, Compound 1 induces the phosphorylation and activation of p38 kinase in a dose dependent manner.

### Example 9

### Compound 1 Induces Phosphorylation of MSK1, an Upstream Target of p38 Kinase

MDA-MB-435 tumor cells were either untreated or treated with 10 nM, 50 nM, 100 nM, 500 nM or 1000 nM of Compound 1 in DMSO for 4 hours. After treatment the cells were lysized and used for Western blot analysis.

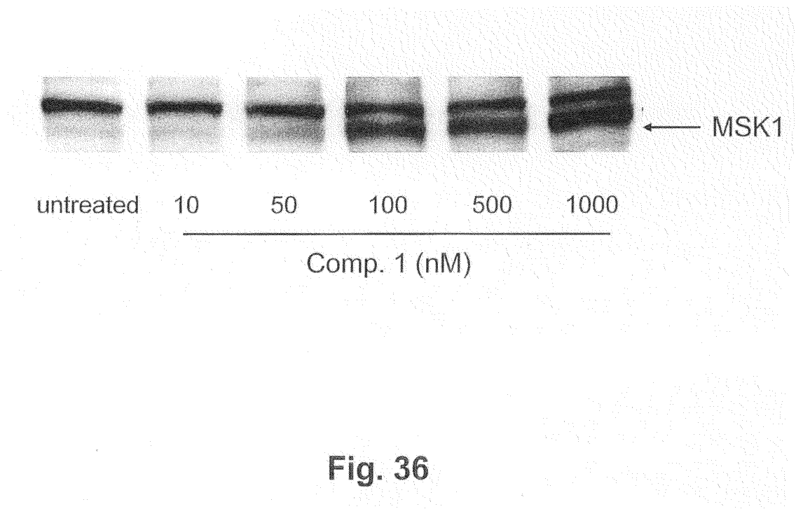

Samples were run on a SDS-PAGE gel and proteins were transferred onto a nitrocellulose membrane by a semi-dry transfer. Nitrocellulose was blocked with 5% skim milk in TBS with 0.5% Tween at room temperature for 1 hour and then probed with anti-p-MSK1 (Thr581) (#9595, Cell Signaling) antibody. The nitrocellulose was then probed with secondary antibodies, HRP-conjugated goat anti-rabbit IgG and HRP-conjugated horse anti-mouse IgG (#7074, #7076, Cell Signaling). LumiGLO reagent, 20× Peroxide (#7003, Cell Signaling) was used to visualize gel (see FIG. 36). As can be seen in FIG. 36, Compound 1 induces the phosphorylation of MSK1 in a dose dependent manner.

### Example 10

### Activation of MSK1 by Compound 1 can be Blocked by a p38 Specific Inhibitor

MDA-MB-435 tumor cells were untreated or treated with 0.1 μM or 0.5 μM of Compound 1 in the presence of 0 μM or 20 μM of p38 inhibitor (SB203580) in DMSO for 0, 30, or 240 minutes. After treatment the cells were lysized and used for Western blot analysis.

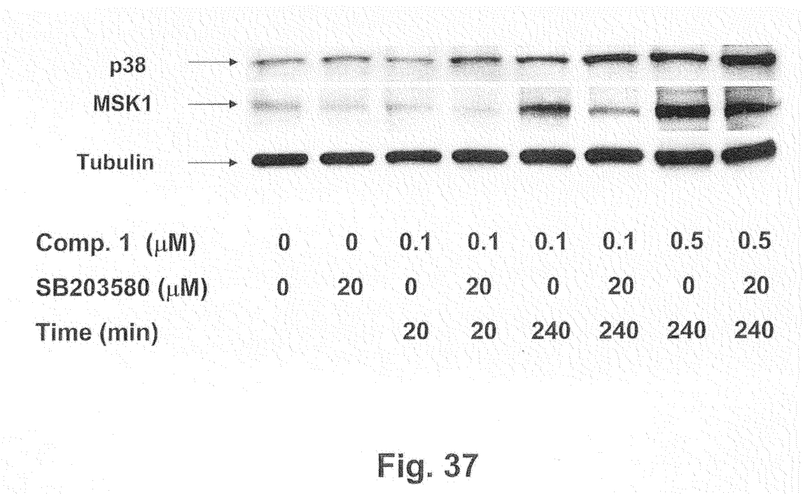

Samples were run on a SDS-PAGE gel and proteins were transferred onto a nitrocellulose membrane by a semi-dry transfer. Nitrocellulose was blocked with 5% skim milk in TBS with 0.5% Tween at room temperature for 1 hour and then probed with anti-p-MSK1 (Thr581) (#9595, Cell Signaling) antibody. Tubulin was used as a normalization control and was probed with Anti-Tubulin (T9026, Sigma) antibody. The nitrocellulose was then probed with secondary antibodies, HRP-conjugated goat anti-rabbit IgG and HRP-conjugated horse anti-mouse IgG (#7074, #7076, Cell Signaling. LumiGLO reagent, 20× Peroxide (#7003, Cell Signaling) was used to visualize gel (see FIG. 37). As can be seen in FIG. 37, Compound 1 induced phosphorylation of MSK1 in MDA-MB-435 tumor cells was blocked by p38 kinase inhibitor SB203580.

### Example 11

### Activation of MAPKAPK-2 by Compound 1 Can be Blocked by a p38 Specific Inhibitor

Tumor cells CV-1 or CEM were untreated or with 0.1 μM or 0.5 μM of Compound 1 in the presence of 0 μM or 20 μM of p38 inhibitor (SB203580) in DMSO for 0, 30, or 240 minutes. After treatment the cells were lysized and used for Western blot analysis.

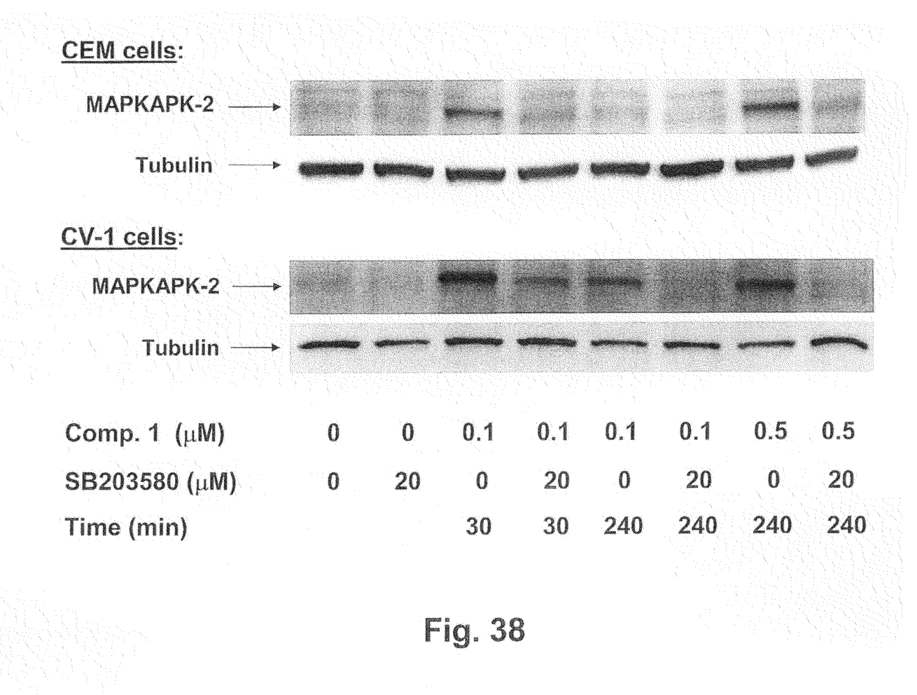

Samples were run on a SDS-PAGE gel and proteins were transferred onto a nitrocellulose membrane by a semi-dry transfer. Nitrocellulose was blocked with 5% skim milk in TBS with 0.5% Tween at room temperature for 1 hour and then probed with anti-p-MAPKAPK-2 (Thr334) (#3041, Cell Signaling) antibody. Tubulin was used as a normalization control and was probed with Anti-Tubulin (T9026, Sigma) antibody. The nitrocellulose was then probed with secondary antibodies, HRP-conjugated goat anti-rabbit IgG and HRP-conjugated horse anti-mouse IgG (#7074, #7076, Cell Signaling. LumiGLO reagent, 20× Peroxide (#7003, Cell Signaling) was used to visualize gel (see FIG. 38). As can be seen in FIG. 38, Compound 1 induced phosphorylation of MAPKAPK-2 in tumor cells CEM and CV-1 was blocked by p38 kinase inhibitor SB203580.

### Example 12

### Activation of p38 Kinase Signaling is Not Required for Compound 1-Induced Apoptosis

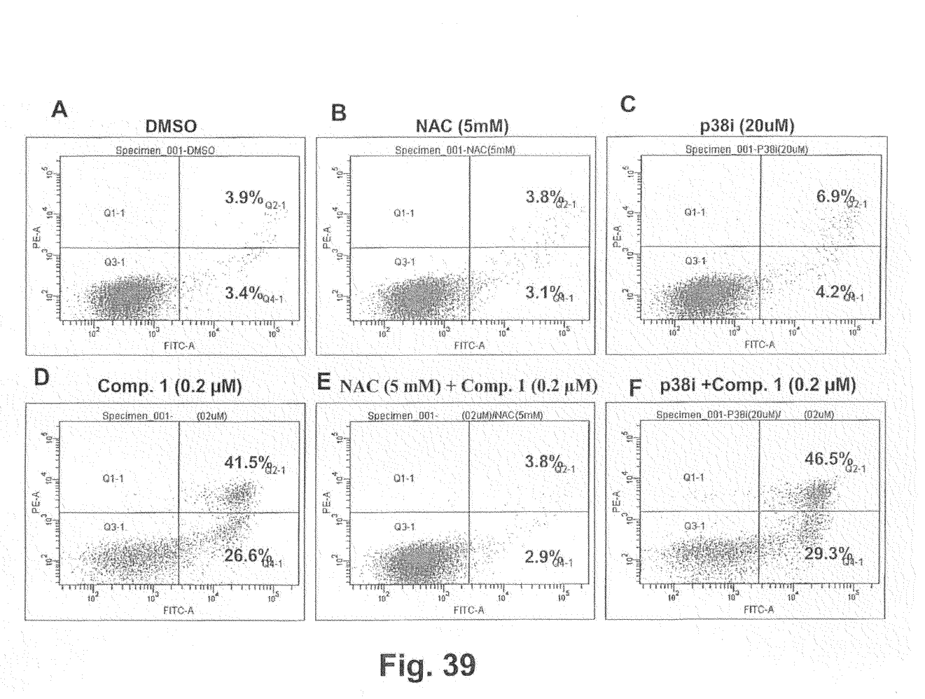

In Example 8, we showed that Compound 1 induces a rapid and sustained activation of p38, a MAP kinase that is associated with the induction of apoptosis in oxidative stressed cells. In this experiment, we determined the affect of a p38 kinase inhibitor on the ability of Compound 1 to induce apoptosis in HSB2 cells (FIG. 39A-F). HSB2 cells were seeded in 6-well plates and pretreated without or with NAC or p38 inhibitor (SB203580, Cat# S8307, Sigma) for 30 minutes and then treated without or with Compound 1 for 20 hours. The cells were harvested and stained with Annexin V and propidium iodide for measuring apoptotic cells by FACS analysis.

Exposure of cells to DMSO, NAC and p38 inhibitor alone had little or no effect on the relative levels of apoptotic cells. When cells were treated with 200 nM Compound 1, a robust increase in the number of both early and late stage apoptotic cells was observed. As expected, NAC treatment completely blocked drug-induced apoptosis. In contrast, the p38 inhibitor had no affect on drug-induced apoptosis. Taken together, these data indicate that p38 is not involved in oxidative stress induced apoptosis by Compound 1.

### Example 13

### Activation of p38 Kinase by Compound 1 Mediates the Induction HSP70

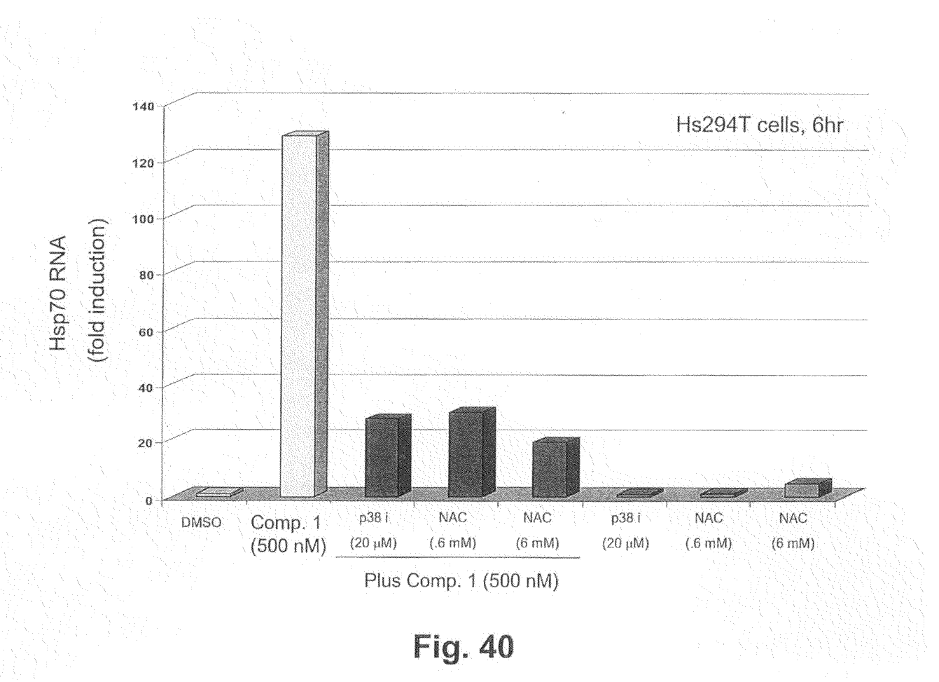

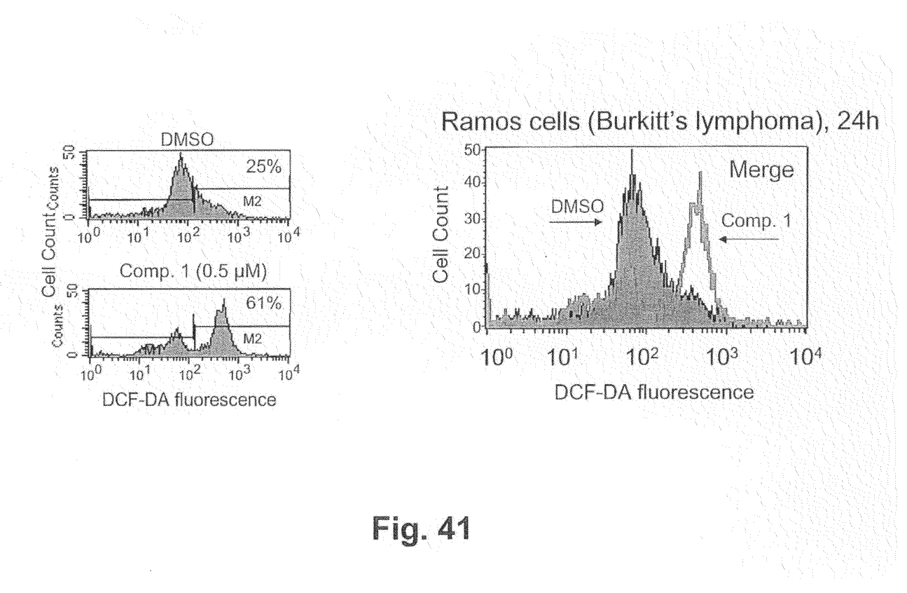

Activation of HSF-1 by oxidative stress has been reported in the literature to require p38 kinase signaling in some cell lines. We therefore examined the effect of inhibiting p38 kinase on the induction of Hsp70 by Compound 1 (FIG. 40). Hs294 T cells were plated for 3 hours and then NAC (f.c. 5 mM) or the p38 inhibitor (f.c 20 uM) (SB 203580, Calbiochem, Cat. No. 559389) was added for 30 minutes prior to treatment with Compound 1 (100 nM). RNA was isolated 6 hours after addition of Compound 1 and QPCR performed as in Example 7. Hsp70 was robustly induced by Compound 1 in Hs294T cells and was completely inhibited by the addition of the antioxidant NAC as expected (see Example 7). As can be seen from FIG. 40, treatment of Compound 1 treated cells with the p38 inhibitor resulted in a robust decrease in Hsp70 induction. These results suggest that oxidative stress activated p38 mediates the induction of Hsp70 by Compound 1.

### Example 14

### Direct Evidence that Compound 1 Induces the Generation of ROS in Drug-Treated Cells

We performed a series of experiments to directly show that Compound 1 induces the generation of ROS in drug-treated cells. In these experiments, we monitored the production of ROS using the cell permeable Carboxy-H₂DCFDA probe. When this probe is oxidized by ROS, it emits a green fluorescence that can be detected using flow cytometry or fluorescent microscopy. Ramos cells were treated with Compound 1 (0.5 μM) for 24 hours or pre-treated with 1.0 mM or 10 mM NAC for 1 hour prior to addition of Compound 1 then washed gently once with warm HBSS/Ca/Mg (GIBCO. 14025). 25 μM carboxy-H₂DCFDA (Invitrogen. C400) working solution was added to cover the cells, and the cells were incubate for 30 minutes at 37° C., protected from light. The cells were gently washed three times in warm HBSS/Ca/Mg. ROS levels, as determined by fluorescence intensity, were determined by flow cytometry (excitation at 495 nm, emission at 529 nm).

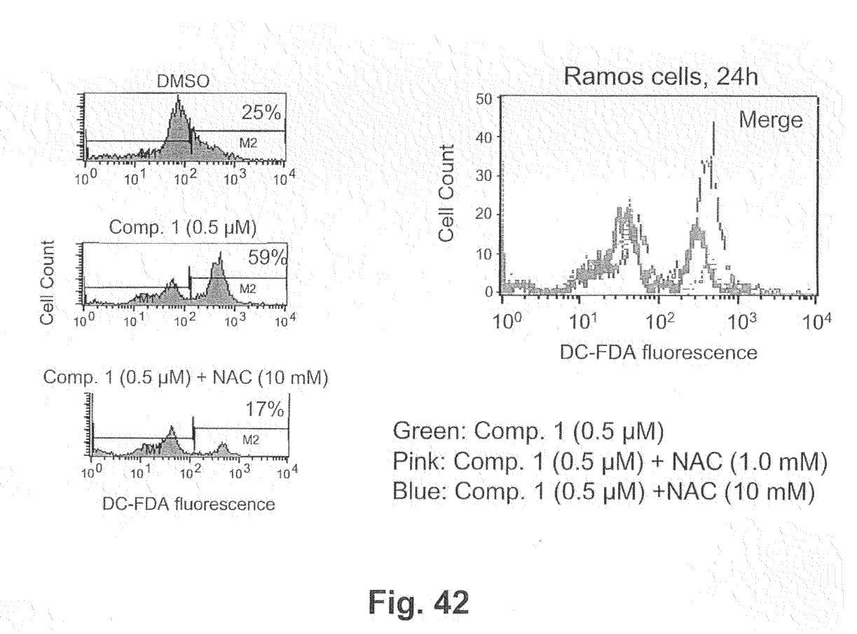

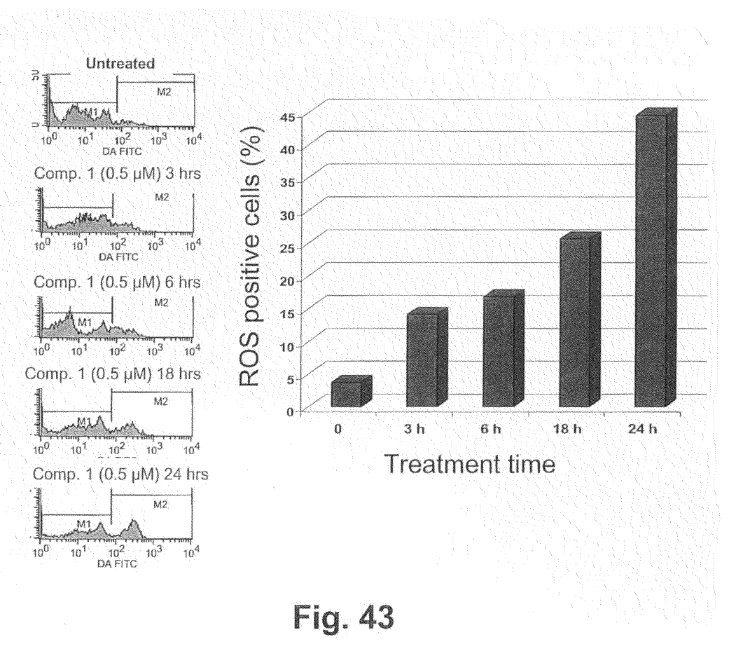

Treatment of Ramos cells for 24 hours with Compound 1 induced the generation of ROS in cells resulting in the oxidation and activation of the Carboxy-H₂DCFDA probe (FIG. 41). Treatment of drug-treated cells with NAC almost completely blocked the generation of ROS by Compound 1 (FIG. 42). Compound 1 induced ROS in a time dependent manner and induction of ROS was observed as early as 3 hours (FIG. 43). These data indicate that Compound 1 induces oxidative stress and ROS generation in drug-treated cells.

### Example 15

### The Free-Radical Scavenger Tiron also Blocks ROS Generation and Hsp70 Induction by Compound 1

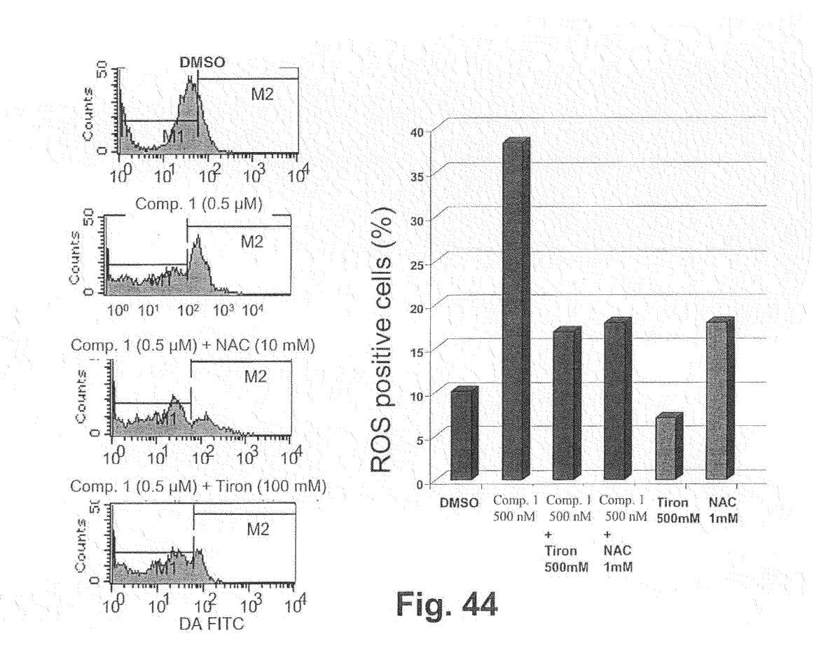

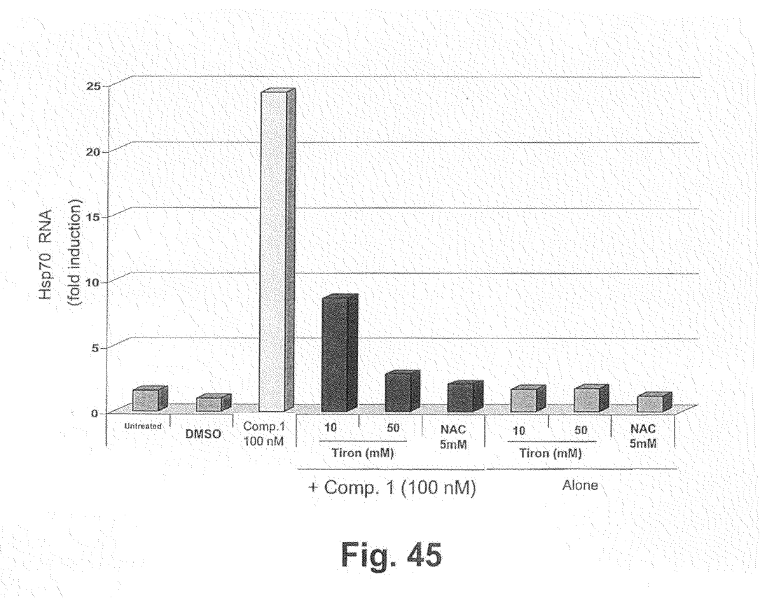

Tiron is a direct scavenger of ROS and is a potent inhibitor of oxidative stress. Ramos cells were pre-treated with 500 mM Tiron 1 hour prior to addition of Compound 1 (0.5 μM) for 24 hours, then measurement of ROS levels was performed as in Example 14. The addition of Tiron to Compound 1-treated cells blocked the ability of Compound 1 to generate ROS (FIG. 44) and induce Hsp70 (FIG. 45). These data demonstrate that multiple antioxidants with differing mechanisms of action can block the activity of Compound 1.

### Example 16

### Compound 1 Enhances the Anti-Cancer Activity of Paclitaxel In Vivo

General Procedure of In Vivo Anti-Tumor Study

The in vivo anti-cancer enhancing effect of novel compounds was assessed in tumor bearing mice using the tumor growth inhibition assay. Tumor cells were implanted by injection of a tumor cell suspension subcutaneously in the flank of a mouse. Treatment of the tumor with a Compound 1 and Paclitaxel begun after the tumor had been established (volume was about 100 mm³). Animals then began a multiple injection schedule where Compound 1 and Paclitaxel were given by IV route of administration. Tumors were measured two times a week. During the course of this assay, animals were monitored daily for signs of toxicity including body weight loss.

A supplemented media was prepared from 50% DMEM/Dulbecco Modified Eagle Medium (High Glucose), 50% RPMI 1640, 10% FBS/Fetal Bovine Serum (Hybridoma Tested; Sterile Filtered), 1% L-Glutamine, 1% Penicillin-Streptomycin, 1% MEM Sodium Pyruvate and 1% MEM Non-Essential Amino Acids. FBS was obtained from Sigma Chemical Co. and other ingredients were obtained from Invitrogen Life Technologies, USA). The supplemental Media was warmed to 37° C. and 50 mL of media was added to a 175 cm tissue culture flask.

The cells used in the assay were MDA-MB-435 Human Breast Carcinoma from the American Type Culture Collection. 1 vial of MDA-MB-435 cells from the liquid nitrogen frozen cell stock was removed. The frozen vial of cells was immediately placed into a 37° C. water bath and gently swirled until thawed. The freeze-vial was wiped with 70% ethanol and cells were immediately pipetted into the 175 cm² tissue culture flask containing supplemented media. The cells were incubated overnight and the media was removed and replaced with fresh supplemented media the next day. The flask was incubated until flask became ˜90% confluent. This took anywhere from 5-7 days.

The flask was washed with 10 mL of sterile room temperature phosphate buffered saline (PBS). The cells were trypsinized by adding 5 mL of warmed Trypsin-EDTA (Invitrogen) to the flask of cells. The cells were then incubated for 2-3 minutes at 37° C. until cells begun to detach from the surface of the flask. An equal volume of supplemented media (5 mL) was added to the flask. All the cells were collected into 50 mL tube, and centrifuged at 1000 RPM for 5 minutes at 20° C. The supernatant was aspirated and the cell pellet was resuspended in 10 mL of supplemented media and the cells were counted. 1-3 million cells/flask were seeded into 5-7 tissue culture flasks (175 cm²). Each flask contained 50 mL of supplemented media. The flasks were incubated until about 90% confluent. The passaging of the cells was repeated until enough cells have been grown for tumor implantation.

The above procedure for trypsinizing and centrifuging the cells were followed. The supernatant was aspirated and the cell pellet was resuspended in 10 mL of sterile PBS and the cells were counted. The cells were centrifuged and then resuspended with appropriate volume of sterile PBS for injection of correct number of cells needed for tumor implantation. In the case of MDA-MB-435, 100 million cells were suspended with 2.0 mL of sterile PBS to a final concentration of 50 million cells/mL in order to inject 5 million cells in 0.1 mL/mouse.

Mice (CD-1 nu/nu) were obtained from Charles River Laboratories: nomenclature: Crl:CD-1-nuBR, Age: 6-8 weeks. The mice were allowed to acclimate for 1 week prior to their being used in an experimental procedure.

Implantation of the MDA-MB-435 tumor cell suspension took place into the corpus adiposum of the female CD-1 nu/nu mouse. This fat body is located in the ventral abdominal viscera of the mouse. Tumor cells were implanted subcutaneously into the fat body located in the right quadrant of the abdomen at the juncture of the os coxae (pelvic bone) and the os femoris (femur). 5 million MDA-MB-435 cells in 0.1 mL of sterile PBS were injected using 27 G (½ inch) needle. MDA-MB-435 tumors developed 2-3 weeks after implantation.

Compound stock solutions were prepared by dissolving the compound in cell-culture-grade DMSO (dimethyl sulfoxide) at the desired concentration. This stock solution in DMSO was sonicated in an ultrasonic water bath until all the powder dissolved.

The Formulation Solvent was prepared as follows: 20% of Cremophore RH40 (Polyoxyl 40 Hydrogenated Castor Oil obtained from BASF corp.) in water was prepared by first heating 100% Cremophore RH40 in a water bath at 50-60° C. until it liquefied and became clear. 10 mL of the 100% Cremophore RH40 aliquoted into a conical centrifuge tube containing 40 mL of sterile water (1:5 dilution of Cremophore RH40). The 20% Cremophore RH40 solution was reheated until it became clear again, and mixed by inverting the tube several times. This 20% Cremophore RH40 solution was stored at room temperature, and was kept for up to 3 months.

Preparation of Dosing Solution for Compound Administration: the Compound stock solution was diluted 1:10 with 20% Cremophore RH40:1) 2.0 mL of 10 mg/mL dosing solution of Compound 1 was prepared by diluting 100 mg/mL Compound Stock solution with 1.8 mL of 20% Cremophore RH40 water solution; and 2) a dosing solution comprising 2.0 mL of 1 mg/mL of Paclitaxel (obtained from Sigma Chemical Co.) and 5 mg/mL of Compound 1 was obtained by mixing 0.1 mL of Compound 1 DMSO stock solution (50 mg/mL) and 0.1 mL of Paclitaxel DMSO stock solution (10 mg/mL) and diluting with 1.8 mL of 20% Cremophore RH40 water solution. The final formulation for the dosing solution was 10% DMSO, 18% Cremophore RH40 and 72% water.

The Dosing Solution (Dosing Volume: 0.01 mL/gram=10 mL/kg) was injected intravenously into the mice bearing MDA-MB-435 human breast tumor. Mice in each treatment group were dosed 3 times a week (Monday, Wednesday, and Friday) for 3 weeks as described in Table 2. There were 5 mice in each group.

TABLE 2

| Group | Dosage |
|---|---|
| 1 | Vehicle |
| 2 | Paclitaxel (5 mg/kg) |
| 3 | Compound 1 (50 mg/kg) |
| 4 | Paclitaxel (5 mg/kg) + Compound 1 (25 mg/kg) |
| 5 | Paclitaxel (5 mg/kg) + Compound 1 (50 mg/kg) |

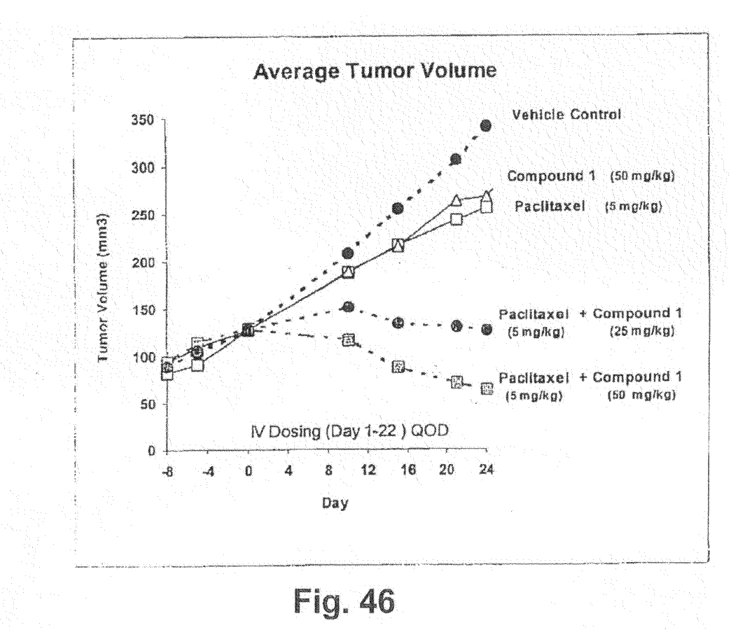

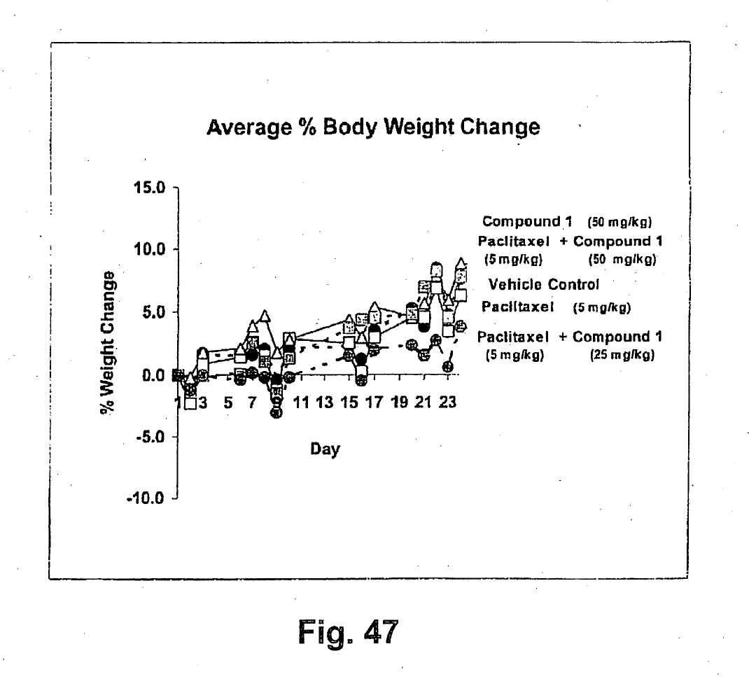

Results: FIG. 46 shows the effects of Compound 1 on enhancing anti-tumor activity of paclitaxel. As can be seen from FIG. 46, Compound 1 significantly enhanced anti-tumor activity of paclitaxel on human breast tumor MDA-MB-435 in nude mice. FIG. 47 shows the effects of Compound 1 and paclitaxel on the body weight of nude mice bearing MDA-MB-435 human breast tumor. As can be seen from FIG. 47, Compound 1 significantly enhanced anti-tumor activity of paclitaxel without increasing toxicity.

### Example 17

### Compound 1 Increased Plasma Levels of Hsp70 in Patients with Cancer

Plasma Hsp70 was measured by a sandwich ELISA kit (Stressgen Bioreagents Victoria, British Columbia, CANADA) according to a modified protocol in house. In brief, Hsp70 in plasma specimens and serial concentrations of Hsp70 standard were captured onto 96-well plate on which anti-Hsp70 antibody was coated. Then captured Hsp70 was detected with a biotinylated anti-Hsp70 antibody followed by incubation with europium-conjugated streptavidin. After each incubation, unbound materials were removed by washing. Finally, antibody-Hsp70 complex was measured by time resolved fluorometry of europium. Concentration of Hsp70 was calculated from a standard curve.

A Phase I trial was conducted for combined administration of Compound 1 and paclitaxel to human subjects with various advanced solid tumors. Compound 1 and paclitaxel were co-administered intravenously over 3 hours once every 3 weeks. Starting doses were 44 milligrams/meter² (mg/m², or 110 micromoles/meter² (μmol/m²)) Compound 1 and 135 mg/m² (158 μmol/m²) paclitaxel. Paclitaxel was then increased to 175 mg/m² (205 μmol/m²), followed by escalation of Compound 1 to establish the maximum tolerated dose based on first cycle toxicity in 3 to 6 patients at each dose level. Pharmacokinetic (PK) studies were performed during cycle 1 using liquid chromatography/mass spectrometry (LC/MS) to measure both Compound 1 and paclitaxel in plasma. Hsp70 was measured in plasma before and after treatment. 35 patients were evaluated at 8 dose levels, including paclitaxel at 135 mg/m² (158 μmol/m²) and Compound 1 at 44 mg/m², and paclitaxel at 175 mg/m² (205 μmol/m²) and Compound 1 at a doses ranging among 44-525 mg/m² (110-1311 μmol/m2). Table 3 shows the eight different doses #1-#8 in mg/m² and μmol/m².

TABLE 3

| | #1 | #2 | #3 | #4 | #5 | #6 | #7 | #8 |
|---|---|---|---|---|---|---|---|---|
| Compound 1, mg/m² | 44 | 44 | 88 | 175 | 263 | 350 | 438 | 525 |
| Compound 1, μmol/m² | 110 | 110 | 220 | 437 | 657 | 874 | 1094 | 1311 |
| Paclitaxel, mg/m² | 135 | 175 | 175 | 175 | 175 | 175 | 175 | 175 |
| Paclitaxel, μmol/m² | 158 | 205 | 205 | 205 | 205 | 205 | 205 | 205 |

No serious side effects specifically attributable to Compound 1 were observed. Paclitaxel dose limiting toxicities occurred in a single patient in each of the top three dose levels (neutropenia, arthralgia, and febrile neutropenia with mucositis) resulting in cohort expansion. Compound 1 exhibited linear PK that was unaffected by paclitaxel dose, and was rapidly eliminated from plasma with terminal-phase half life of 0.94±0.23 hours (h) and total body clearance of 28±8 Liters/hour/meter² (L/h/m²). Its apparent volume of distribution was comparable to total body water (Vₛₛ 23±16 L/m²). Paclitaxel PK appeared to be moderately dependent on the Compound 1 dose, as indicated by a significant trend toward decreasing clearance, and increase in peak plasma concentration and Vₛₛ, but without affecting the terminal phase half-life. These observations are consistent with competitive inhibition of paclitaxel hepatic metabolism. Increased toxicity at higher dose levels was consistent with a moderate increase in systemic exposure to paclitaxel. Induction of Hsp70 protein in plasma was dose dependent, peaking between about 8 hours to about 24 hours after dosing.

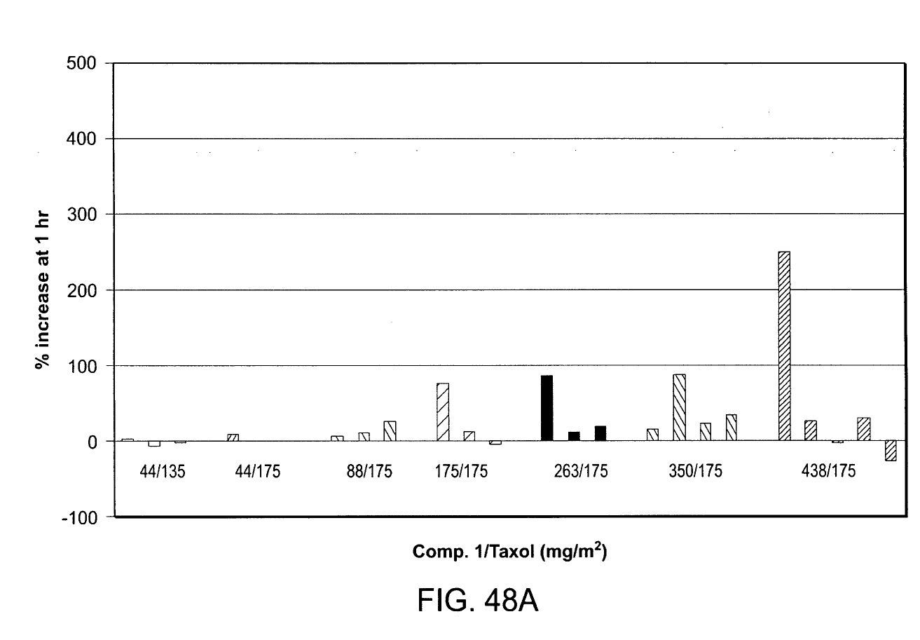

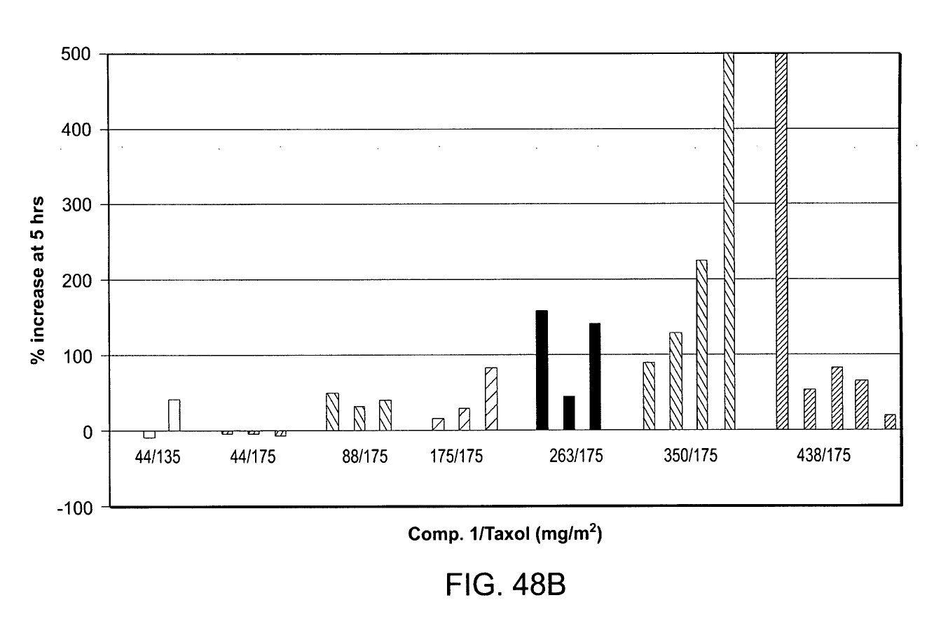

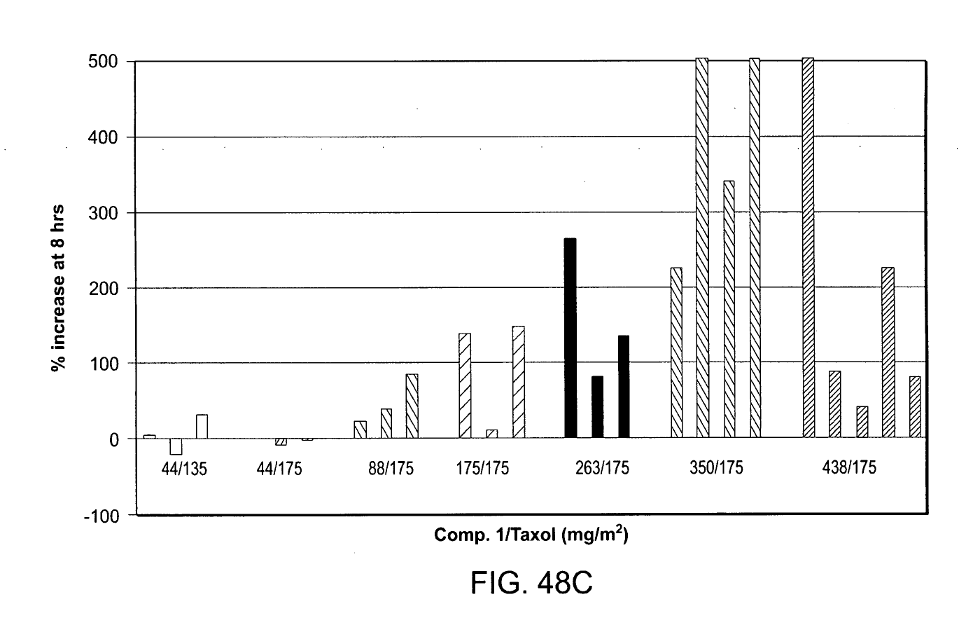

FIGS. 48A, 48B, and 48C are bar graphs showing the percent increase in Hsp70 plasma levels associated with administration of the Compound 1/paclitaxel combination therapy at 1 hour (FIG. 48A), 5 hours (FIG. 48B), and 8 hours (FIG. 48C) after administration. Significant rises in Hsp70 levels occurred for at least one patient at the 88 mg/m² (220 μmol/m²) Compound 1 dose, where Hsp70 levels nearly doubled in a percent increase of about 90%. At the 175 mg/m2 (437 μmol/m²) Compound 1 dose, Hsp70 concentrations more than doubled in two patients; at the 263 mg/m² (657 μmol/m²) Compound 1 dose, Hsp70 concentrations roughly doubled in two patients and increased by more than 250% in a third patient; at the 350 mg/m² (874 μmol/m²) Compound 1 dose, Hsp70 concentrations increased more than 200% in all patients and increased by as much as 500% in two patients; at the 438 mg/m² (1094 μmol/m²) Compound 1 dose, Hsp70 concentrations roughly doubled in two patients, increased by over 2005 in one patient, and increased by as much as 500% in another patient.

Thus, the combination of Compound 1 and a taxane dramatically increased plasma Hsp70 levels in patients, giving significant increases for patients at a combined paclitaxel dose of 175 mg/m2 (205 μMol/m²) and Compound 1 doses ranging from 88 through 438 mg/M2 (220-1094 μmol/m²). Moreover, the combination was well-tolerated, with adverse events consistent with those expected for paclitaxel alone.

### Example 18

### Treatment with Compound 1 and Paclitaxel Increased Time to Progression in Stage 1V Melanoma Patients

A phase II clinical trial was conducted in patients that had Stage 1V metastatic melanoma patients. The patients were given either a weekly treatment regimen of Compound 1 and paclitaxel combined or paclitaxel alone, and time to progression was assessed.

A total of 81 people with Stage 1V melanoma were tested in a randomized trial with ratios of 2:1, Compound (1)+paclitaxel (53 people): paclitaxel alone (28 people). The dosages administered were 213 mg/m² Compound (1), 80 mg/m² paclitaxel, and the dosage regimen was 3 weekly doses per each 4 week cycle. Patients were treated until progression of the disease. Patients who progressed on paclitaxel alone were given the option to crossover to Compound (1)+paclitaxel and were treated until progression. The tumor assessments were performed at baseline, Cycle 2, and every other Cycle thereafter.

Table 4 shows the Kaplier Meyer estimates of the Time to Progression of the disease (Efficacy Sample):

TABLE 4

| | compound (1) + | | |
|---|---|---|---|
| | Paclitaxel | Paclitaxel | |
| | (n = 50) | (n = 27) | p-value* |
| Time to Progression(days)25^{th} percentile (95%confidence interval (CI))Median (95% CI)75^{th} percentile (95% CI) | 54.0 (49.0, 95.0)134.0 (86.0, 217.0)273.0 (168.0, 331.0) | 49.0 (29.0, 52.0)56.0 (49.0, 105.0)106.0 (61.0, 218.0) | 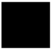 |

The p-value is from a log-rank test

The study results indicated a statistically significant (i.e., p-value less than 0.05) increase in the time to progression in patients that were treated with Compound 1 plus paclitaxel compared with patients treated with paclitaxel alone.

Table 5 shows the best overall response per Response Evaluation Criteria In Solid Tumors (RECIST) (Efficacy Sample)

TABLE 5

| | compound (1) + | | |
|---|---|---|---|
| | Paclitaxel | Paclitaxel | |
| | (n = 50) | (n = 27) | p-value* |
| Best Overall Response | | | |
| Complete Response (CR) | 1 (2.0%) | 0 | |
| Partial Response (PR) | 7 (14.0%) | 1 (3.7%) | |
| Stable Disease (SD) | 25 (50.0%) | 10 (37%) | |
| Progressive Disease (PD) | 17 (34.0%) | 16 (59.3%) | |
| Two-Sided Fisher's Exact | | | |
| Test | | | |
| CR + PR (95% CI) | 16.0% (7.2%,29.1%) | 3.7% (0.1%,19.0%) | 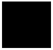 |

As can be seen from Table 5 compounds of the present invention in combination with paclitaxel show a significant improvement over paclitaxel alone. Specifically compounds of the present invention in combination with paclitaxel showed one patent with a complete response, 14% of patients with partial response and 50% of the patients had stable disease compared with paclitaxel alone which only showed 1 patient with partial response and 37% of the patients with stable disease. In addition, 59.3% of patients treated with paclitaxel alone had progressive disease whereas only 34.0% of patients treated with the combination of Compound 1 and paclitaxel had progressive disease.

Tables 6 and 7 show the relative treatment results of Compound 1 in combination with Paclitaxel compared with Paclitaxel alone and other currently used treatments for melanoma. As can be seen from Tables 6 and 7 the number of days to progression of the disease is greatly enhanced for Compound 1 in combination with paclitaxel compared with paclitaxel alone. In addition the time to progression benefit is much better than any single-agent therapy and much better than all but one combination therapy.

The combination therapy, cisplatin vinblastine dacarbazine IL-2 and IFN, which had a longer time to progression than Compound 1 in combination with paclitaxel, however, has severe side effects and requires patients to be hospitalized for administration of the combination. Conversely, Compound 1 in combination with paclitaxel only showed a mild increase in the side effects over paclitaxel alone. None of the side effects were sever enough to cause any patients to discontinue treatment with Compound 1 in combination with Paclitaxel during the trial.

TABLE 6

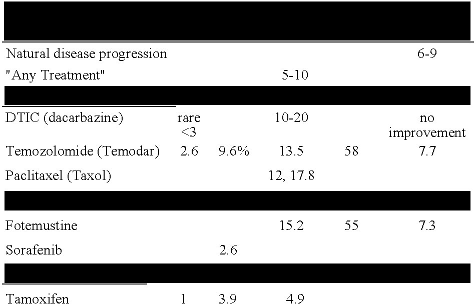

TABLE 7

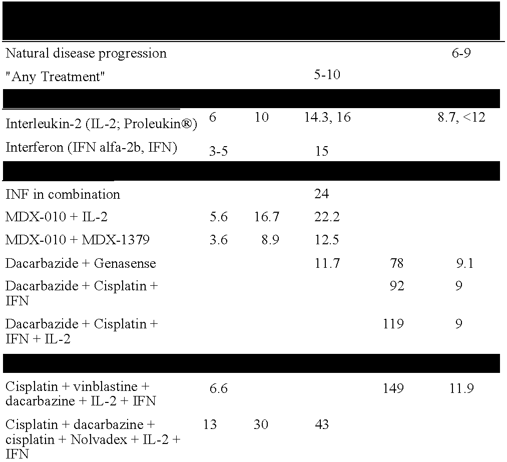

### Example 19

### Induction of Accumulation of ROS in Cells Shortly After Elesclomol Treatment

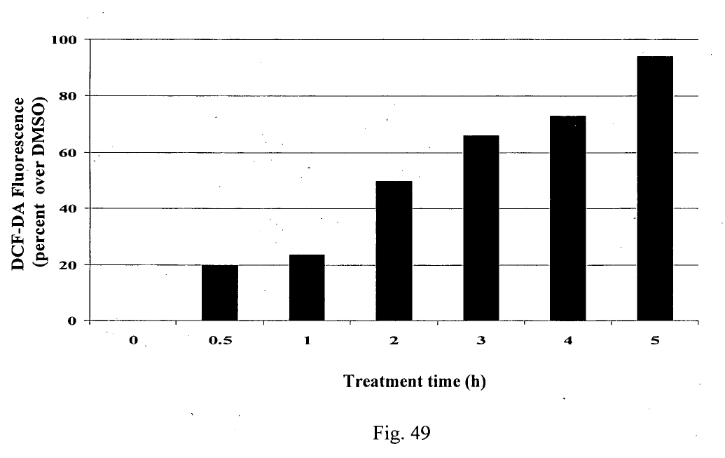

Ramos cells were treated with elesclomol (500 nM) for the indicated times then washed gently once with warm HBSS/Ca/Mg (Invitrogen Carlsbad, Calif. Cat# 14025). 25 μM carboxy-H2DCFDA (Invitrogen Cat #C400) working solution was added to cover the cells and they were incubated for 30 minutes at 37° C., protected from light. The cells were gently washed three times in warm HBSS/Ca/Mg. ROS levels (fluorescence intensity) were determined by flow cytometry (excitation at 495 nm, emission at 529 nm) using a Becton Dickinson FACSCalibur equipped with CellQuest Pro software. Data is plotted as percent of elesclomol-treated cells with DCFDA fluorescence relative to DMSO (control) treated cells (see FIG. 49). As shown in FIG. 49, these results demonstrate that elesclomol induces accumulation of ROS in cells shortly after drug treatment.

### Example 20

### Induction of Hsp70 in Cells Shortly After Elesclomol Treatment

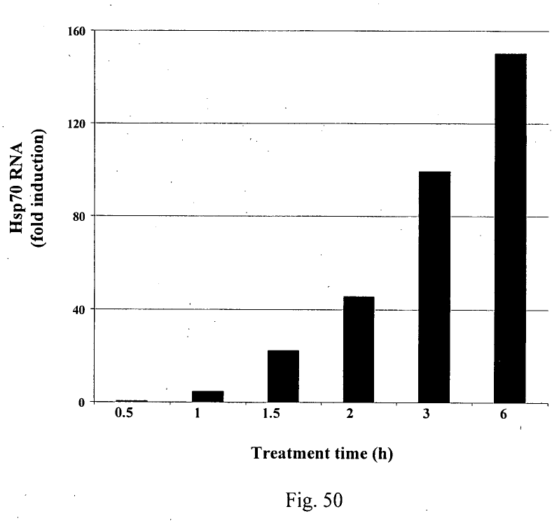

Ramos cells were treated with elesclomol (100 nM) or DMSO for the indicated times. Following treatment, total RNA was isolated using the QiaShredder and RNeasy kits (Qiagen, Valencia, Calif.) according to the manufacturer's instructions. 500 ng of RNA from each sample was used for cDNA synthesis using iScript cDNA Synthesis Kit (Biorad, Hercules, Calif. Cat# 170-8891). 1 ul of cDNA mixture is used with the iQ SYBR Green Supermix (Biorad, Cat# 170-8882) and quantitative PCR performed in an iCycler (Biorad) using standard protocols. QPCR for each sample/time point was performed in duplicate. The following primers for inducible human Hsp70 and gapdh were used: Hsp70-F3 5′-AAG-GAC-ATC-AGC-CAG-AAC-AAG-CG-3′, Hsp70-R3 5′-AAG-AAG-TCC-TGC-AGC-AGC-TTC-TGC-3′, Gapdh-F2 5′-AAG-GTC-GGA-GTC-AAC-GGA-TTT-GGT-3′, Gapdh-R2 5′-CAT-GGT-TCA-CAC-CCA-TGA-CGA-ACA-3′. Ct values are obtained from the iCycler program and used in an equation (2^{-ΔΔC}T) for determining the amount of target relative to gapdh expression. This compares the relative expression of Hsp70 to the corresponding levels of gapdh in each sample. Duplicate wells were averaged and fold of Hsp70 RNA induction relative to DMSO control was plotted, as shown in FIG. 50. These results demonstrate that elesclomol induces Hsp70 induction concomitantly with ROS accumulation, at early times post-drug treatment.

### Example 21

### Activation of Apoptotic Markers Cytochrome c and Caspase 3 Shortly After Elesclomol Treatment

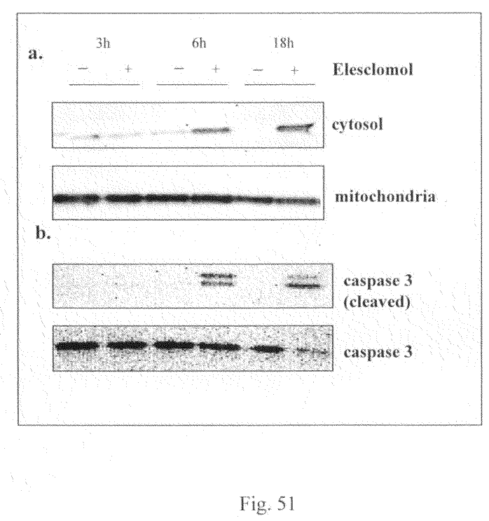

Ramos cells were plated for 3 hours then treated with 100 nM elesclomol for the indicated times. To determine cytochrome c levels in the cytoplasm and mitochondria (a) Ramos cells were fractionated using the method detailed in (Hase et al, Journal of Biol. Chem. Vol. 277, pp. 46950-58, 2002) and western blots were probed with Cytochrome C primary antibody (BD Pharmingen, clone 7H8.2; C12). All Western blots were probed with secondary antibodies conjugated to HRP (Cell Signaling) and visualized using Lumiglo (Cell Signaling) peroxide reagents. Images were captured from the Kodak Image Station 440. For Caspase 3 analysis (b), whole cell lysates were isolated using 1× cell lysis buffer (Cell Signaling, Danvers, Mass.) plus protease inhibitors (Calbiochem, San Diego, Calif.). 30 ug of protein was run on a 4-12% Nupage gel (Invitrogen) and immunoblotted using standard procedures. The blots were probed with Caspase 3 antibody (9662) and cleaved Caspase 3 primary antibodies (9661) both from Cell Signaling. The results are summarized in FIG. 51. As shown in FIG. 51, these results demonstrate that the apoptotic markers cytochrome c and caspase 3 are activated starting at 6 hours post elesclomol treatment.

The relevant teachings of all publications, patents and patent applications cited herein that have not explicitly been incorporated herein by reference, are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

1. A compound, or pharmaceutically acceptable salt thereof, that increases oxidative stress in a cell, wherein the compound increases p38 activity, provided that the compound is not a compound disclosed in the patents and patent applications listed in Table 1.

2. The compound of claim 1, wherein the compound increases cellular expression of Hsp70.

3. The compound of claim 2, wherein the compound increases membrane-bound Hsp70 of the cell.

4. The compound of claim 3, wherein the Hsp70 on the surface of the cell attracts natural killer cells.

5. The compound of claim 2, wherein the compound increases the amount of Hsp70 secreted by the cell.

6. The compound of claim 5, wherein the Hsp70 secreted by the cell attracts natural killer cells to the cell.

7. The compound of claim 1, wherein the compound increases the activity of ASK1.

8. The compound of claim 7, wherein the compound disrupts the association of ASK1 and thioredoxin.

9. The compound of claim 1, wherein the compound increases the efficacy of an additional anti-cancer therapy.

10. The compound of claim 9, wherein the compound increases the efficacy of paclitaxel or a paclitaxel analog.

11. A method of treating cancer in a subject, comprising the steps of:
a) administering to the subject an anti-cancer therapy; and
b) administering to the subject a compound that increases oxidative stress in the cancer cells and increases the activity of p38, provided that the compound is not a compound having the following structural formula:

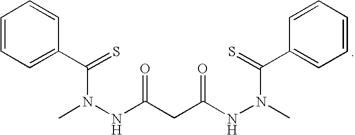

12. The method of claim 11, wherein the compound increases cellular expression of Hsp70.

13. The method of claim 12, wherein the compound increases membrane-bound Hsp70 of the cell.

14. The method of claim 13, wherein the Hsp70 on the surface of the cell attracts natural killer cells.

15. The method of claim 12, wherein the compound increases the amount of Hsp70 secreted by the cell.

16. The method of claim 15, wherein the Hsp70 secreted by the cell attracts natural killer cells to the cell.

17. The method of claim 11, wherein the compound increases the activity of ASK1.

18. The method of claim 17, wherein the compound disrupts the association of ASK1 and thioredoxin.

19. The method of claim 11, wherein the subject is human.

20. The method of claim 11, wherein the anti-cancer therapy is a chemotherapeutic agent.

21. The method of claim 20, wherein the chemotherapeutic agent is paclitaxel or a paclitaxel analog.

22. The method of claim 11, wherein the cancer is melanoma, renal cell carcinoma, pancreatic cancer, or bladder cancer.

23. A method of identifying a compound that has synergistic anticancer activity with a taxane, comprising the steps of:
a) determining Hsp70 expression in the cell before contacting the cell with a test compound;
b) contacting the cell with the test compound; and
c) determining Hsp70 expression in the cell after contact with the test compound, wherein increased levels of Hsp70 expression in the cell after contact with the test compound indicates that the compound has synergistic anticancer activity with a taxane.

24. The method of claim 23, further comprising the steps of:
d) determining the activity of p38 before the cell is contacted with the test compound; and
e) determining the activity of p38 after the cell has been contacted with the test compound, wherein increased activity of p38 in the cell after contact with the test compound indicates that the compound has synergistic anticancer activity with a taxane.

25. A pharmaceutical composition, comprising a pharmaceutically acceptable carrier and a compound of claim 1, or pharmaceutically acceptable salt thereof.
